SEQUENCE LISTING (55 sequences)

SEQ ID NO: 1 - protein, 13 aa, Homo Sapiens
EDPQGDAAQKTDT

SEQ ID NO: 2 - protein, 10 aa, Homo Sapiens
LEALKENGGA

SEQ ID NO: 3 - protein, 6 aa, Homo Sapiens
NTEGLQ

SEQ ID NO: 4 - protein, 11 aa, Homo Sapiens
GGHLDQQVEEF

SEQ ID NO: 5 - protein, 9 aa, Homo Sapiens
DQNVEELKG

SEQ ID NO: 6 - protein, 11 aa, Homo Sapiens
SVQESQVAQQA

SEQ ID NO: 7 - protein, 12 aa, Homo Sapiens
TAKDALSSVQES

SEQ ID NO: 8 - protein, 7 aa, Homo Sapiens
TVGSLAG

SEQ ID NO: 9 - protein, 10 aa, Homo Sapiens
DEVKEQVAEV

SEQ ID NO: 10 - protein, 13 aa, Homo Sapiens
VGTSAAPVPSDNH

SEQ ID NO: 11 - protein, 15 aa, Homo Sapiens
VTEPISAESGEQVER

SEQ ID NO: 12 - protein, 12 aa, Homo Sapiens
SEETKENEGFTV

SEQ ID NO: 13 - protein, 10 aa, Homo Sapiens
SEETKENEGF

SEQ ID NO: 14 - protein, 17 aa, Homo Sapiens
SEETKENEGFTVTAEGK

SEQ ID NO: 15 - protein, 7 aa, Homo Sapiens
HWESASL

SEQ ID NO: 16 - protein, 7 aa, Homo Sapiens
TLEIPGN

SEQ ID NO: 17 - protein, 12 aa, Homo Sapiens
GSESGIFTNTKE

SEQ ID NO: 18 - protein, 11 aa, Homo Sapiens
SEADHEGTHST

SEQ ID NO: 19 - protein, 11 aa, Homo Sapiens
SESGIFTNTKE

SEQ ID NO: 20 - protein, 13 aa, Homo Sapiens
DEAGSEADHEGTH

SEQ ID NO: 21 - protein, 10 aa, Homo Sapiens
GDFLAEGGGV

SEQ ID NO: 22 - protein, 12 aa, Homo Sapiens
DEAGSEADHEGT

SEQ ID NO: 23 - protein, 14 aa, Homo Sapiens
GSESGIFTNTKESS

SEQ ID NO: 24 - protein, 15 aa, Homo Sapiens
DEAGSEADHEGTHST

SEQ ID NO: 25 - protein, 13 aa, Homo Sapiens
SESGIFTNTKESS

SEQ ID NO: 26 - protein, 17 aa, Homo Sapiens
DEAGSEADHEGTHSTKR

SEQ ID NO: 27 - protein, 9 aa, Homo Sapiens
NRGDSTFES

SEQ ID NO: 28 - protein, 8 aa, Homo Sapiens
FLAEGGGV

SEQ ID NO: 29 - protein, 11 aa, Homo Sapiens
SYNRGDSTFES

SEQ ID NO: 30 - protein, 11 aa, Homo Sapiens
NRGDSTFESKS

SEQ ID NO: 31 - protein, 8 aa, Homo Sapiens
STFESKSY

SEQ ID NO: 32 - protein, 7 aa, Homo Sapiens
DFLAEGG

SEQ ID NO: 33 - protein, 11 aa, Homo Sapiens
EGDFLAEGGGV

SEQ ID NO: 34 - protein, 10 aa, Homo Sapiens
EGDFLAEGGG

SEQ ID NO: 35 - protein, 17 aa, Homo Sapiens
MADEAGSEADHEGTHST

SEQ ID NO: 36 - protein, 9 aa, Homo Sapiens
DFLAEGGGV

SEQ ID NO: 37 - protein, 9 aa, Homo Sapiens
DSTFESKSY

SEQ ID NO: 38 - protein, 16 aa, Homo Sapiens
FTSSTSYNRGDSTFES

SEQ ID NO: 39 - protein, 14 aa, Homo Sapiens
DSGEGDFLAEGGGV

SEQ ID NO: 40 - protein, 20 aa, Homo Sapiens
SYKMADEAGSEADHEGTHST

SEQ ID NO: 41 - protein, 10 aa, Homo Sapiens
DFLAEGGGVR

SEQ ID NO: 42 - protein, 19 aa, Homo Sapiens
YKMADEAGSEADHEGTHST

SEQ ID NO: 43 - protein, 8 aa, Homo Sapiens
DFLAEGGG

SEQ ID NO: 44 - protein, 15 aa, Homo Sapiens
ADSGEGDFLAEGGGV

SEQ ID NO: 45 - protein, 12 aa, Homo Sapiens
NRGDSTFESKSY

SEQ ID NO: 46 - protein, 13 aa, Homo Sapiens
GSGSGWSSSRGPY

SEQ ID NO: 47 - protein, 19 aa, Homo Sapiens
LLGLPGPPDVPDHAAYHPF

SEQ ID NO: 48 - protein, 8 aa, Homo Sapiens
LDDDLEHQ

SEQ ID NO: 49 - protein, 9 aa, Homo Sapiens
IGEIKEETT

SEQ ID NO: 50 - protein, 17 aa, Homo Sapiens
LDDDLEHQGGHVLDHGH

SEQ ID NO: 51 - protein, 14 aa, Homo Sapiens
SKETIEQEKQAGES

SEQ ID NO: 52 - protein, 13 aa, Homo Sapiens
KETIEQEKQAGES

SEQ ID NO: 53 - protein, 12 aa, Homo Sapiens
ETIEQEKQAGES

SEQ ID NO: 54 - protein, 12 aa, Homo Sapiens
GPPASSPAPAPK

SEQ ID NO: 55 - protein, 19 aa, Homo Sapiens
QLGLPGPPDVPDHAAYHPF

(19) United States
(12) Patent Application Publication
(10) Pub. No.: US 2016/0018413 A1
(43) Pub. Date: Jan. 21, 2016

(54) Methods of Prognosing Preeclampsia

(71) Applicants: THE BOARD OF TRUSTEES OF THE LELAND STANFORD JUNIOR UNIVERSITY, Palo Alto, CA (US); RIKEN, Wako-shi (JP)

(72) Inventors: Bruce Xuefeng Ling, Palo Alto, CA (US); Ting Yang, San Jose, CA (US); Atul J. Butte, Menlo Park, CA (US); Linda Liu Miller, Philadelphia, PA (US); Qiaojun Wen, Stanford, CA (US); Guojun Sheng, Saitama (JP); Cantas Alev, Saitama (JP)

(21) Appl. No.: 14/773,300

(22) PCT Filed: Mar. 13, 2014
(86) PCT No.: PCT/US14/26124

§ 371 Date: Sep. 4, 2015

Related U.S. Application Data
(60) Provisional application No. 61/783,450, filed on Mar. 14, 2013.

(51) Int. Cl. G01N 33/68; H01J 49/00

(52) U.S. Cl. CPC G01N 33/6893; H01J 49/0027; G01N 2800/368; G01N 2800/52

(57) ABSTRACT

Preeclampsia peptide biomarkers are provided. Also provided are methods for using these biomarkers, including in prognosing or diagnosing preeclampsia in a pregnant individual by detecting these biomarkers in a sample from the pregnant individual. Reagents, devices and kits thereof that find use in practicing the subject methods are also provided.

### CROSS REFERENCE TO RELATED APPLICATIONS

This application is a 371 of International Application No. PCT/US2014/026124 filed Mar. 13, 2014, which claims priority to the filing date of the U.S. Provisional Patent Application Ser. No. 61/783,450 filed Mar. 14, 2013; the full disclosure of which is herein incorporated by reference.

### FIELD OF THE INVENTION

This invention pertains to peptide biomarkers for prognosing preeclampsia.

### BACKGROUND OF THE INVENTION

Preeclampsia is a serious multisystem complication of pregnancy with adverse effects for mothers and babies. If unaddressed, preeclampsia can lead to eclampsia, i.e. seizures that are not related to a preexisting brain condition. The incidence of the disorder is around 5-8% of all pregnancies in the U.S. and worldwide, and the disorder is responsible for 18% of all maternal deaths in the U.S. The causes and pathogenesis of preeclampsia remain uncertain, and the diagnosis relies on nonspecific laboratory and clinical signs and symptoms that occur late in the disease process, sometimes making the diagnosis and clinical management decisions difficult. Earlier and more reliable disease diagnosing, prognosing and monitoring will lead to more timely and personalized preeclampsia treatments and significantly advance our understanding of preeclampsia pathogenesis. The present invention addresses these issues.

### SUMMARY OF THE INVENTION

Preeclampsia peptide biomarkers are provided. Also provided are methods for using these biomarkers, including in prognosing or diagnosing preeclampsia in a pregnant individual by detecting these biomarkers in a sample from the pregnant individual. Reagents, devices and kits thereof that find use in practicing the subject methods are also provided.

In some aspects of the invention, a preeclampsia peptide representation, e.g. a preeclampsia peptide signature or score, is obtained for a pregnant individual. In some embodiments, the preeclampsia peptide representation is obtained by obtaining a blood sample from the individual; measuring the abundance of a panel of preeclampsia peptide biomarkers in the sample; and evaluating the abundance of peptides. In some embodiments, the sample is obtained from the individual at or before gestational week 34. In certain embodiments, the sample is collected from the individual at or before gestational week 25. In some embodiments, the measuring comprises mass spectrometry. In certain embodiments, evaluating the abundance of peptides comprises summing the amount of each preeclampsia peptide across MS fractions, normalizing to the sum of the amounts of all preeclampsia peptides across all MS fractions to obtain a score for each peptide, and analyzing the scores, e.g. by predictive analysis of microarrays (PAM), to arrive at a single preeclampsia representation, e.g. a preeclampsia signature or score. In some embodiments, a report is provided, providing the preeclampsia peptide representation, and in some instances, a reference to which it can be compared, e.g. to make a preeclampsia prognosis or diagnosis.

In some aspects of the invention, a preeclampsia peptide representation for an individual that is obtained, e.g. as disclosed above and herein, is employed to provide a preeclampsia prognosis or diagnosis to a pregnant individual. In some embodiments, the method comprises comparing the preeclampsia peptide representation to a reference, and providing a diagnosis or prognosis based on the comparison. In some embodiments, the prognosis or diagnosis is provided by providing a report.

In some embodiments, the panel comprises 5 or more peptides derived from polypeptides selected from the polypeptides in Tables 2 and 3, i.e., the group consisting of alpha-1-antitrypsin (A1AT), apolipoprotein A-I (APO-A1), apolipoprotein A-IV (APO-A4), apolipoprotein C-III (APO-C3), apolipoprotein E (APO-E), apolipoprotein L 1 (APO-L1), complement component 3 (C3), complement component 4A (C4A), fibrinogen alpha chain (FGA), hornerin (HRNR), inter-alpha-trypsin inhibitor heavy chain H4 (ITIH4), kininogen-1 (KNG-1), thymosin beta-4-like protein 1 (TMSB4), and zyxin (ZYX). In some embodiments, the peptides include the peptides listed in Table 3 or Table 4, i.e. the one or more peptides derived from the A1AT polypeptide is EDPQGDAAQKTDT (SEQ ID NO:1); the one or more peptides derived from the APO-A1 polypeptide is LEALKENGGA (SEQ ID NO:2); the one or more peptides derived from the APO-A4 polypeptide is NTEGLQ (SEQ ID NO:3), GGHLDQQVEEF (SEQ ID NO:4), or DQNVEELKG (SEQ ID NO:5); the one or more peptides derived from the APO-C3 polypeptide is SVQESQVAQQA (SEQ ID NO:6) or TAKDALSSVQES (SEQ ID NO:7); the one or more peptides derived from the APO-E polypeptide is TVGSLAG (SEQ ID NO:8), DEVKEQVAEV (SEQ ID NO:9), or VGTSAAPVPSDNH (SEQ ID NO:10); the one or more peptides derived from the APO-L1 polypeptide is VTEPISAESGEQVER (SEQ ID NO:11); the one or more peptides derived from the C3 polypeptide is SEETKENEGFTV (SEQ ID NO:12), SEETKENEGF (SEQ ID NO:13), SEETKENEGFTVTAEGK (SEQ ID NO:14), or HWESASL (SEQ ID NO:15); the one or more peptides derived from the C4A polypeptide is TLEIPGN (SEQ ID NO:16); the one or more peptides derived from the FGA polypeptide is GSESGIFTNTKE (SEQ ID NO:17), SEADHEGTHST (SEQ ID NO:18), SESGIFTNTKE (SEQ ID NO:19), DEAGSEADHEGTH (SEQ ID NO:20), GDFLAEGGGV (SEQ ID NO:21), DEAGSEADHEGT (SEQ ID NO:22), GSESGIFTNTKESS (SEQ ID NO:23), DEAGSEADHEGTHST (SEQ ID NO:24), SESGIFTNTKESS (SEQ ID NO:25), DEAGSEADHEGTHSTKR (SEQ ID NO:26), NRGDSTFES (SEQ ID NO:27), FLAEGGGV (SEQ ID NO:28), SYNRGDSTFES (SEQ ID NO:29), NRGDSTFESKS (SEQ ID NO:30), STFESKSY (SEQ ID NO:31), DFLAEGG (SEQ ID NO:32), EGDFLAEGGGV (SEQ ID NO:33), EGDFLAEGGG (SEQ ID NO:34), MADEAGSEADHEGTHST (SEQ ID NO:35), DFLAEGGGV (SEQ ID NO:36), DSTFESKSY (SEQ ID NO:37), FTSSTSYNRGDSTFES (SEQ ID NO:38), DSGEGDFLAEGGGV (SEQ ID NO:39), SYKMADEAGSEADHEGTHST (SEQ ID NO:40), DFLAEGGGVR (SEQ ID NO:41), YKMADEAGSEADHEGTHST (SEQ ID NO:42), DFLAEGGG (SEQ ID NO:43), ADSGEGDFLAEGGGV (SEQ ID NO:44), or NRGDSTFESKSY (SEQ ID NO:45); the one or more peptides derived from the HRNR polypeptide is GSGSGWSSSRGPY (SEQ ID NO:46); the one or more peptides derived from the ITIH4 polypeptide is LLGLPGPPDVPDHAAYHPF (SEQ ID NO:47); the one or more peptides derived from the KNG-1 polypeptide is LDDDLEHQ (SEQ ID NO:48), IGEIKEETT (SEQ ID NO:49), or LDDDLEHQGGHVLDHGH (SEQ ID NO:50); the one or more peptides derived from the TMSB4 polypeptide is SKETIEQEKQAGES (SEQ ID NO:51), KETIEQEKQAGES (SEQ ID NO:52), or ETIEQEKQAGES (SEQ ID NO:53); and the one or more peptides derived from the ZYX polypeptide is GPPASSPAPAPK (SEQ ID NO:54).

In certain embodiments, the panel comprises 6 or more peptides derived from the polypeptides listed in Table 4, i.e., A1AT, APO-L1, FGA, ITIH4, KNG-1, and TMSB4. In some such embodiments, the panel comprises 6 or more peptides selected from the group consisting of EDPQGDAAQKTDT (SEQ ID NO:1), VTEPISAESGEQVER (SEQ ID NO:11), GSESGIFTNTKE (SEQ ID NO:17), SEADHEGTHST (SEQ ID NO:18), SESGIFTNTKE (SEQ ID NO:19), DEAGSEADHEGTH (SEQ ID NO:20), GDFLAEGGGV (SEQ ID NO:21), DEAGSEADHEGT (SEQ ID NO:22), GSESGIFTNTKESS (SEQ ID NO:23), DEAGSEADHEGTHST (SEQ ID NO:24), SESGIFTNTKESS (SEQ ID NO:25), DEAGSEADHEGTHSTKR (SEQ ID NO:26), NRGDSTFES (SEQ ID NO:27), FLAEGGGV (SEQ ID NO:28), SYNRGDSTFES (SEQ ID NO:29), NRGDSTFESKS (SEQ ID NO:30), STFESKSY (SEQ ID NO:31), DFLAEGG (SEQ ID NO:32), EGDFLAEGGGV (SEQ ID NO:33), EGDFLAEGGG (SEQ ID NO:34), MADEAGSEADHEGTHST (SEQ ID NO:35), DFLAEGGGV (SEQ ID NO:36), DSTFESKSY (SEQ ID NO:37), FTSSTSYNRGDSTFES (SEQ ID NO:38), DSGEGDFLAEGGGV (SEQ ID NO:39), SYKMADEAGSEADHEGTHST (SEQ ID NO:40), DFLAEGGGVR (SEQ ID NO:41), YKMADEAGSEADHEGTHST (SEQ ID NO:42), DFLAEGGG (SEQ ID NO:43), ADSGEGDFLAEGGGV (SEQ ID NO:44), NRGDSTFESKSY (SEQ ID NO:45), LLGLPGPPDVPDHAAYHPF (SEQ ID NO:47), LDDDLEHQ (SEQ ID NO:48), IGEIKEETT (SEQ ID NO:49), LDDDLEHQGGHVLDHGH (SEQ ID NO:50), SKETIEQEKQAGES (SEQ ID NO:51), KETIEQEKQAGES (SEQ ID NO:52), and ETIEQEKQAGES (SEQ ID NO:53). In certain embodiments, the panel comprises the peptides listed in the 19-peptide panel in Table 4, i.e. EDPQGDAAQKTDT (SEQ ID NO:1), VTEPISAESGEQVER (SEQ ID NO:11), GSESGIFTNTKESS (SEQ ID NO:23), GSESGIFTNTKE (SEQ ID NO:17), SESGIFTNTKE (SEQ ID NO:19), SYKMADEAGSEADHEGTHST (SEQ ID NO:40), DEAGSEADHEGTHST (SEQ ID NO:24), DEAGSEADHEGT, SEADHEGTHST (SEQ ID NO:18), ADSGEGDFLAEGGGV (SEQ ID NO:44), DSGEGDFLAEGGGV (SEQ ID NO:39), DFLAEGGGV (SEQ ID NO:36), NRGDSTFESKSY (SEQ ID NO:45), NRGDSTFES (SEQ ID NO:27), DSTFESKSY (SEQ ID NO:37), LLGLPGPPDVPDHAAYHPF (SEQ ID NO:47), LDDDLEHQ (SEQ ID NO:48), IGEIKEETT (SEQ ID NO:49), and SKETIEQEKQAGES (SEQ ID NO:51).

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 shows the serum concentrations of sFlt-1 (left) and PIGF (right) as a function of the gestation. For either PE (red) or control (green) data points, a loess curve was fitted to represent the overall trend of biomarker serum abundance as a function of gestation.

FIG. 2A-2C demonstrates the process of PE serum peptide biomarker discovery and validation. (FIG. 2A) Study outline. (FIG. 2B) Heatmap display of the differential (SAM algorithm, q<0.05) serum peptide biomarkers. The rows on the heatmap represent the 52 peptides derived from 14 different proteins with each column of that row representing a different sample from subjects with PE (red) and control (green) subjects. Within PE or control groups, the samples are ordered by gestational age from early to late weeks. (FIG. 2C) Predictor panel discovery by PAM was performed with all the peptide identifications found by LC/MS. In training (black line) and cross-validation (blue line), decreasing the threshold (lower x-axis) resulted in an increase in the number of peptides (inserted upper x-axis) that were used for classification and calculation of the classification error (y-axis). The blue dashed lines represents the variance estimate of predicted error. This led to the discovery of a set of 120 peptides with lowest possible classification error and a minimal practical set of 19 peptides (on the right).

FIG. 3 shows the PAM predictive analysis of the 19-peptide biomarker panel differentiating PE from control samples. PAM prediction was performed with training data from PE (training, n=21; testing, n=10) and control (training, n=21; testing, n=10) samples evaluated with the biomarker panel. Samples are partitioned by the true class (upper) and predicted class (lower). The classification results from training and test sets are shown as 2 by 2 contingency tables, calculating the percentage of classifications that agreed with clinical diagnosis.

FIG. 4 shows the diagnosis of PE from control with serum biomarkers. Left panel: estimated PE scores were computed from the PE serum peptide panel PAM model as a function of the gestational weeks; right panel: the log sFlt-1/PIGF serum concentration ratio was plotted as a function of the gestational weeks. Red indicates known PE cases; green indicates known healthy pregnancy controls. For either PE or control sample category, a loess curve was fitted to represent the overall trend of biomarker scoring as a function of gestational age.

### DETAILED DESCRIPTION OF THE INVENTION

Preeclampsia peptide biomarkers are provided. Also provided are methods for using these biomarkers, including in prognosing or diagnosing preeclampsia in a pregnant individual by detecting these biomarkers in a sample from the pregnant individual. Reagents, devices and kits thereof that find use in practicing the subject methods are also provided. These and other objects, advantages, and features of the invention will become apparent to those persons skilled in the art upon reading the details of the compositions and methods as more fully described below.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Preeclampsia Peptide Panels

In some aspects of the invention, preeclampsia peptide biomarkers and panels of preeclampsia peptide biomarkers are provided, which panels may be used in the prognosis, diagnosis, and/or treatment of a subject for preeclampsia. By “preeclampsia” or “pre-eclampsia” it is meant the multisystem complication of pregnancy characterized by high blood pressure, e.g. 140/90 mm/Hg or higher, and protein in the urine (proteinuria). By a “peptide” it is meant an amino acid sequence of approximately 50 amino acids or less. By a “preeclampsia peptide” or a ‘preeclampsia peptide biomarker” it is meant a peptide that is differentially represented in a biological sample, e.g. a blood or serum sample, from an individual that will develop or has developed preeclampsia as compared to an individual that will not develop preeclampsia. In other words, the peptide biomarker is present in different amounts in a sample from individual that will develop/has developed preeclampsia as compared to a healthy individual. By the term “will develop preeclampsia” it is meant that a subject has a high probability of developing preeclampsia within at least about 4 weeks, within at least about 3 week, within at least about 2 weeks, within at least about 1 week. The terms “subject,” “individual,” “host,” and “patient,” are used interchangeably herein and refer to any mammalian subject for whom diagnosis, treatment, or therapy is desired, particularly humans.

The subject preeclampsia peptide panels are based in part on the discovery of 52 peptides listed in Table 2 that are differentially represented in subjects that will develop or have developed preeclampsia as compared to individuals that will not develop preeclampsia. As such, in some instances, panels of preeclampsia peptide biomarkers are provided, where the panels comprise 2 or more peptides listed in Table 3 or Table 4, i.e., EDPQGDAAQKTDT (SEQ ID NO:1), LEALKENGGA (SEQ ID NO:2), NTEGLQ (SEQ ID NO:3), GGHLDQQVEEF (SEQ ID NO:4), DQNVEELKG (SEQ ID NO:5), SVQESQVAQQA (SEQ ID NO:6), TAKDALSSVQES (SEQ ID NO:7), TVGSLAG (SEQ ID NO:8), DEVKEQVAEV (SEQ ID NO:9), VGTSAAPVPSDNH (SEQ ID NO:10), VTEPISAESGEQVER (SEQ ID NO:11), SEETKENEGFTV (SEQ ID NO:12), SEETKENEGF (SEQ ID NO:13), SEETKENEGFTVTAEGK (SEQ ID NO:14), HWESASL (SEQ ID NO:15), TLEIPGN (SEQ ID NO:16), GSESGIFTNTKE (SEQ ID NO:17), SEADHEGTHST (SEQ ID NO:18), SESGIFTNTKE (SEQ ID NO:19), DEAGSEADHEGTH (SEQ ID NO:20), GDFLAEGGGV (SEQ ID NO:21), DEAGSEADHEGT (SEQ ID NO:22), GSESGIFTNTKESS (SEQ ID NO:23), DEAGSEADHEGTHST (SEQ ID NO:24), SESGIFTNTKESS (SEQ ID NO:25), DEAGSEADHEGTHSTKR (SEQ ID NO:26), NRGDSTFES (SEQ ID NO:27), FLAEGGGV (SEQ ID NO:28), SYNRGDSTFES (SEQ ID NO:29), NRGDSTFESKS (SEQ ID NO:30), STFESKSY (SEQ ID NO:31), DFLAEGG (SEQ ID NO:32), EGDFLAEGGGV (SEQ ID NO:33), EGDFLAEGGG (SEQ ID NO:34), MADEAGSEADHEGTHST (SEQ ID NO:35), DFLAEGGGV (SEQ ID NO:36), DSTFESKSY (SEQ ID NO:37), FTSSTSYNRGDSTFES (SEQ ID NO:38), DSGEGDFLAEGGGV (SEQ ID NO:39), SYKMADEAGSEADHEGTHST (SEQ ID NO:40), DFLAEGGGVR (SEQ ID NO:41), YKMADEAGSEADHEGTHST (SEQ ID NO:42), DFLAEGGG (SEQ ID NO:43), ADSGEGDFLAEGGGV (SEQ ID NO:44), NRGDSTFESKSY (SEQ ID NO:45), GSGSGWSSSRGPY (SEQ ID NO:46), LLGLPGPPDVPDHAAYHPF (SEQ ID NO:47), LDDDLEHQ (SEQ ID NO:48), IGEIKEETT (SEQ ID NO:49), LDDDLEHQGGHVLDHGH (SEQ ID NO:50), SKETIEQEKQAGES (SEQ ID NO:51), KETIEQEKQAGES (SEQ ID NO:52), ETIEQEKQAGES (SEQ ID NO:53), and GPPASSPAPAPK (SEQ ID NO:54). In some instances, the panel comprises 2 of the subject peptides. In some instances, the panel comprises 3, 4, or 5 or more peptides, for example, 6, 7, 8, 9, or 10 or more peptides, in some instances, 11, 12, 13, 14, 15, 16, 17, 18, 19 or 20 or more peptides, e.g., 20, 25, 30, 35, 40, 45, or 50 or more peptides, e.g. the 52 peptides disclosed in Table 3 and Table 4.

In certain instances, the peptide panel comprises a subset of the subject peptides, for example, 10, 11, 12, 13, 14, 15, 16, 17, 18 or 19 of the subject peptides e.g. 10, 11, 12, 13, 14, 15, 16, 17, 18 or 19 of the 19 peptides provided in Table 4, i.e. EDPQGDAAQKTDT (SEQ ID NO:1), VTEPISAESGEQVER (SEQ ID NO:11), GSESGIFTNTKESS (SEQ ID NO:23), GSESGIFTNTKE (SEQ ID NO:17), SESGIFTNTKE (SEQ ID NO:19), SYKMADEAGSEADHEGTHST (SEQ ID NO:40), DEAGSEADHEGTHST (SEQ ID NO:24), DEAGSEADHEGT (SEQ ID NO:22), SEADHEGTHST (SEQ ID NO:18), ADSGEGDFLAEGGGV (SEQ ID NO:44), DSGEGDFLAEGGGV (SEQ ID NO:39), DFLAEGGGV (SEQ ID NO:36), NRGDSTFESKSY (SEQ ID NO:45), NRGDSTFES (SEQ ID NO:27), DSTFESKSY (SEQ ID NO:37), LLGLPGPPDVPDHAAYHPF (SEQ ID NO:47), LDDDLEHQ (SEQ ID NO:48), IGEIKEETT (SEQ ID NO:49), and SKETIEQEKQAGES (SEQ ID NO:51). The working examples provide an example of a preeclampsia peptide panel that may be used in the subject methods. Other preeclampsia peptide panels may be readily identified by the ordinarily skilled artisan by, for example, employing a statistical feature selection process, e.g. as known in the art or described herein. For example, additional preeclampsia peptide panels may be identified using the shrunken centroid algorithm called predictive analysis of microarrays (PAM) (Tibshirani et al. (2002) Diagnosis of multiple cancer types by shrunken centroids of gene expression. Proc Natl Acad Sci USA 98:5116-5121). PAM is a multivariate analysis algorithm used to identify differentially expressed features, e.g. proteins or genes, for biomarker analysis. As another example, additional preeclampsia peptide panels may be identified by combining genetic algorithm (GA) and all paired (AP) support vector machine (SVM) methods for preeclampsia classification analysis. Predictive features are automatically determined, e.g. through iterative GA/SVM, leading to very compact sets of non-redundant preeclampsia-relevant peptides with the optimal classification performance. As will be recognized by the ordinarily skilled artisan, different panels, or classifier sets, may harbor only modest overlapping peptide features, but have similar levels of accuracy.

As demonstrated in Tables 2 and 3, the peptides of the subject preeclampsia peptide panels are derived from 1 of 14 different polypeptides: alpha-1-antitrypsin (A1AT), apolipoprotein A-I (APO-A1), apolipoprotein A-IV (APO-A4), apolipoprotein C-III (APO-C3), apolipoprotein E (APO-E), apolipoprotein L 1 (APO-L1), complement component 3 (C3), complement component 4A (C4A), fibrinogen alpha chain (FGA), hornerin (HRNR), inter-alpha-trypsin inhibitor heavy chain H4 (ITIH4), kininogen-1 (KNG-1), thymosin beta-4-like protein 1 (TMSB4), and zyxin (ZYX). These polypeptides are representative of 3 canonical biological processes: acute inflammatory and defense responses (A1AT, APO-L1, FGA, ITIH4, KNG1), lipid metabolism (APO-A4, APO-C3, APO-E, APO-L1), and the activation of the complement and coagulation responses (A1AT, C3, C4A, FGA). As such, in some instances, the panels of preeclampsia peptides comprise peptides that are representative of 3 canonical biological processes: acute inflammatory and defense responses (A1AT, APO-L1, FGA, ITIH4, KNG1), lipid metabolism (APO-A4, APO-C3, APO-E, APO-L1), and the activation of the complement and coagulation responses (A1AT, C3, C4A, FGA). In certain instances, the panels of preeclampsia peptides comprise peptides derived from two or more polypeptides selected from the group consisting of A1AT, APO-A1, APO-A4, APO-C3, APO-E, APO-L1, C3, C4A, FGA, HRNR, ITIH4, KNG-1, TMSB4, and ZYX. In some instances, the panels of preeclampsia peptides comprise peptides derived from 2 of these polypeptides. In some instances, the panels of preeclampsia peptides comprise peptides derived from 3, 4, or 5 or more polypeptides, e.g. from 6, 7, 8, 9, or 10 or more polypeptides, e.g. 11, 12, 13, or 14 polypeptides, e.g. the 14 polypeptides disclosed in Tables 2 and 3. In certain instances, the peptide panel comprises peptides derived from the 6 polypeptides provided in Table 4, i.e. A1AT, APO-L1, FGA, ITIH4, KNG-1, and TMSB4.

### Methods of Use

As indicated above, the subject preeclampsia peptide biomarkers and panels of preeclampsia peptide biomarkers may be used in the prognosis, diagnosis, and/or treatment of a subject for preeclampsia. To apply the subject preeclampsia peptide panels in such applications, a preeclampsia peptide representation in a sample from the subject is determined. By a “preeclampsia peptide representation,” it is meant the representation in a biological sample of the peptides that make up a subject preeclampsia panel. A preeclampsia peptide representation may be determined by, for example, detecting the abundance, or amount, or level, of preeclampsia peptide(s) in the sample, e.g., a panel of preeclampsia peptides, and evaluating the detected abundance of peptide in the sample to arrive at the preeclampsia peptide representation. As such, in some aspects of the invention, methods are provided for determining a preeclampsia peptide representation for an individual, comprising obtaining a biological sample from an individual, detecting the abundance of peptide(s) for a preeclampsia peptide panel in the sample, and evaluating the detected abundance of peptide in the sample to obtain a preeclampsia peptide representation.

The term “biological sample” encompasses a variety of sample types obtained from an organism and can be used in a diagnostic or monitoring assay. The term encompasses blood and other liquid samples of biological origin or cells derived therefrom and the progeny thereof. The term encompasses samples that have been manipulated in any way after their procurement, such as by treatment with reagents, solubilization, or enrichment for certain components. The term encompasses a clinical sample, and also includes cell supernatants, cell lysates, serum, plasma, biological fluids, and tissue samples. Clinical samples for use in the methods of the invention may be obtained from a variety of sources, particularly blood samples. Once a sample is obtained, it can be used directly, frozen, or maintained in appropriate culture medium for short periods of time. Typically the samples will be from human patients, although animal models may find use, e.g. equine, bovine, porcine, canine, feline, rodent, e.g. mice, rats, hamster, primate, etc. Any convenient tissue sample that demonstrates the differential representation in a patient with preeclampsia of the one or more preeclampsia markers disclosed herein may be evaluated in the subject methods. Typically, a suitable sample source will be derived from fluids into which the molecular entity of interest, i.e. the peptide, has been released. Sample sources of particular interest include blood samples or preparations thereof, e.g., whole blood, or serum or plasma, and urine. A sample volume of blood, serum, or urine between about 2 μl to about 2,000 μl is typically sufficient for determining the level of a preeclampsia peptide. Generally, the sample volume will range from about 10 μl to about 1,750 μl, from about 20 μl to about 1,500 μl, from about 40 μl to about 1,250 μl, from about 60 μl to about 1,000 μl, from about 100 μl to about 900 μl, from about 200 μl to about 800 μl, from about 400 μl to about 600 μl. In many embodiments, a suitable initial source for the human sample is a blood sample. As such, the sample employed in the subject assays is generally a blood-derived sample. The blood derived sample may be derived from whole blood or a fraction thereof, e.g., serum, plasma, etc., where in some embodiments the sample is derived from blood, allowed to clot, and the serum separated and collected to be used to assay.

The subject sample may be treated in a variety of ways so as to enhance detection of the preeclampsia peptides. For example, where the sample is blood, the red blood cells may be removed from the sample (e.g., by centrifugation) prior to assaying. Such a treatment may serve to reduce the non-specific background levels of detecting the level of a preeclampsia peptide. As another example, the sample may be purified by removing proteins, nucleic acids, and the like, e.g. by liquid chromatography, e.g. HPLC, to obtain a sample that is substantially pure in naturally occurring peptides. Detection of a preeclampsia peptide may also be enhanced by concentrating the sample using procedures well known in the art (e.g. acid precipitation, alcohol precipitation, salt precipitation, hydrophobic precipitation, filtration (using a filter which is capable of retaining molecules greater than 30 kD, e.g. Centrim 30™), affinity purification). In some embodiments, the pH of the test and control samples will be adjusted to, and maintained at, a pH which approximates neutrality (i.e. pH 6.5-8.0). Such a pH adjustment will prevent complex formation, thereby providing a more accurate quantitation of the level of marker in the sample. In some embodiments, e.g. where the sample is a urine sample, the pH of the sample is adjusted and the sample is concentrated in order to enhance the detection of the marker.

The subject sample is typically obtained from the individual during the second or third trimester of gestation. By “gestation” it is meant the duration of pregnancy in a mammal, i.e. the period of development in the uterus from conception until birth. The time interval of a gestation plus two weeks, i.e. to the last menstrual period, is called the gestation period. Human gestation can be divided into three trimesters, each three months long. The first trimester is from the last menstrual period to the 13th week, the second trimester is from the 14th to 27th week, and the third trimester is from the 28th week to 42 weeks. A subject sample may be obtain early in gestation, for example, on or before 34 weeks of gestation, e.g. on or before week 25 of gestation, e.g. at weeks 20-25 of gestation, at weeks 26-34 of gestation, at weeks 30-34 weeks of gestation. The subject sample may be obtained late in gestation, for example, after 34 weeks of gestation, e.g. at week 35, week 36, week 37, week 38, week 39, week 40, week 41, or week 42.

In certain embodiments, the sample is a serum or serum-derived sample. Any convenient methodology for producing a fluid serum sample may be employed. In many embodiments, the method employs drawing venous blood by skin puncture (e.g., finger stick, venipuncture) into a clotting or serum separator tube, allowing the blood to clot, and centrifuging the serum away from the clotted blood. The serum is then collected and stored until assayed. In some instances, the obtaining comprises drawing the sample from the subject. In other instances, the obtaining comprises receiving a sample from a practitioner, where the practitioner has drawn the sample from the individual. Once the patient derived sample is obtained, the sample is assayed to detect the level of preeclampsia peptide(s) in the sample.

The amount, i.e., abundance of preeclampsia peptide(s) in the sample may be detected by any convenient method for detecting peptide in a biological sample. For example, Mass Spectrometry (MS) may be employed. In MS, a sample (which may be solid, liquid, or gas) is ionized; the ions are separated according to their mass-to-charge ratio, e.g. by magnetic sector, by radio frequencies (RF) quadrupole field, by time of flight (TOF), etc.; the ions are dynamically detected by some mechanism capable of detecting energetic charged particles, and the signal is processed into the spectra of the masses of the particles of that sample. In some instances, tandem mass spectrometry (MS/MS or MS²) may be employed, for example, to determine the sequences of the peptides separated by MS. For example, a first mass analyzer isolates one peptide from many entering a mass spectrometer. A second mass analyzer then stabilizes the peptide ions and promotes their fragmentation, e.g. by collision-induced dissociation (CID), electron capture dissociation (ECD), electron transfer dissociation (ETD), infrared multiphoton dissociation (IRMPD), blackbody infrared radiative dissociation (BIRD), electron-detachment dissociation (EDD), surface-induced dissociation (SID), etc. A third mass analyzer then sorts the fragments produced from the peptides. For example, a sample may be applied to an LTQ ion trap mass spectrometer equipped with a Fortis tip mounted nano-electrospray ion source, and the fraction scanned with a mass range of 400-2000 m/z. This first MS scan is followed by two data-dependent scans of the two most abundant ions observed in the first full MS scan. Tandem MS can also be done in a single mass analyzer over time, as in a quadrupole ion trap. In some instances, MS is combined with other technologies, e.g. multiple reaction monitoring (MRM) is coupled with stable isotope dilution (SAD) mass spectrometry (MS), which allowed quantitative assays for peptides to be performed with minimum restrictions and the ease of assembling multiple peptide detections in a single measurement. Other methods for detecting peptides in a sample by MS and measuring the abundance of peptides in a sample are well known in the art; see, e.g. the teachings in US 2010/0163721, the full disclosure of which is incorporated herein by reference.

Alternatively, non-MS based-methods for measuring the abundance of one or more peptides in a sample may also be employed. For example, immune-based methods, e.g. ELISA, western blotting, flow cytometry, immunohistochemistry, etc. may be employed. In such methods, antibodies that are specific for the preeclampsia peptide marker(s) of interest but not the polypeptide(s) from which they were derived are used to detect the peptide marker(s) and their abundance. Typically, such antibodies will be specific for a domain created by the cleavage event that generated the peptide, i.e., the antibodies will be cleavage site-specific antibodies. Antibodies that are specific to the polypeptide(s) and not the peptide marker(s) may also be used, which serve as negative control(s).

The resultant data provides information regarding the abundance in the sample of each of the peptides that have been probed, wherein the information is in terms of whether or not the peptide is present and, typically, at what level, and wherein the data may be both qualitative and quantitative. As such, where detection is qualitative, the methods provide a reading or evaluation, e.g., assessment, of whether or not the target peptide(s) is present in the sample being assayed. In yet other embodiments, the methods provide a quantitative detection of whether the target peptide (s) is present in the sample being assayed, i.e., an evaluation or assessment of the actual amount or relative abundance of the target peptide(s) in the sample being assayed. In such embodiments, the quantitative detection may be absolute or, if the method is a method of detecting two or more different peptides in a sample, relative. As such, the term “quantifying” when used in the context of quantifying a target peptide in a sample can refer to absolute or to relative quantification. Absolute quantification may be accomplished by inclusion of known concentration(s) of one or more control peptide(s) and referencing the detected level of the target peptide(s) with the known control peptide(s) (e.g., through generation of a standard curve). Alternatively, relative quantification can be accomplished by comparison of detected levels or amounts between two or more different target peptide(s) to provide a relative quantification of each of the two or more different peptide(s), e.g., relative to each other.

Once the levels of the preeclampsia peptides have been measured, the measurement(s) may be evaluated in any of a number of ways to obtain a preeclampsia peptide representation. For example, the preeclampsia peptide measurements may be analyzed to produce a preeclampsia peptide representation that is a preeclampsia profile. As used herein, a “preeclampsia profile” is the normalized level of one or more preeclampsia peptides in a patient sample, for example, the normalized level of serological peptide concentrations in a patient sample. A profile may be generated by any of a number of methods known in the art. For example, the level of each peptide may be determined by summing up the amount of peptide across MS/MS fractions, and normalized relative to the abundance of a selected housekeeping gene, e.g. ABL1, GAPDH, or PGK1, or to the total intensity value of all peptides found in the sample.

As another example, the preeclampsia peptide measurements may be analyzed to generate a preeclampsia peptide representation that is a preeclampsia signature. By a “preeclampsia signature” it is meant a single metric value that represents the weighted expression levels (e.g. serological peptide concentrations) of the subject panel of preeclampsia peptides in a sample, where the weighted levels are defined by the dataset from which the sample was obtained. A preeclampsia signature for a sample may be calculated by any of a number of methods known in the art for calculating biomarker signatures. For example, the levels of each of the one or more preeclampsia peptide markers in a sample may summed across MS/MS fractions and normalized, e.g. as described above for generating a preeclampsia profile. The normalized expression levels for each peptide marker is then weighted, e.g. using a multivariate analysis algorithm, e.g. PAM, by multiplying the normalized level to a weighting factor, or “weight”, to arrive at weighted expression levels for each of the one or more peptides. The weighted levels are then totaled and in some cases averaged to arrive at a single weighted level for the panel of preeclampsia peptides analyzed. The weighting factor, or weight, may be determined by any statistical machine learning methodology, for example, predictive analysis of microarrays (PAM), principle component analysis (PCA), linear regression, support vector machines (SVMs), applying the dataset from which the sample was obtained, i.e. the “testing dataset” to obtain the weight values. For example, the analyte level of each preeclampsia peptide may be log₂ transformed and weighted either as 1 (for those markers that are increased in level in preeclampsia) or −1 (for those markers that are decreased in level in preeclampsia), and the ratio between the sum of increased peptides as compared to decreased peptides determined to arrive at a preeclampsia signature.

As another example, the preeclampsia peptide measurements may be analyzed to produce a preeclampsia peptide representation that is a preeclampsia score. Like a preeclampsia signature, a “preeclampsia score” is a single metric value that represents the sum of the weighted levels of the preeclampsia peptides in a sample. A preeclampsia score may be determined by methods very similar to those described above for a preeclampsia signature, e.g. the levels of each of the one or more preeclampsia peptides in a sample may be summed across MS/MS fractions and normalized, e.g. as described above for generating a preeclampsia profile; the normalized expression levels for each peptide is then weighted, e.g. using a multivariate analysis algorithm, e.g. PAM, PCA, SVMs, etc., by multiplying the normalized level to a weighting factor, or “weight”, to arrive at weighted levels for each of the one or more peptides; and the weighted levels are then totaled and in some cases averaged to arrive at a single weighted level for the one or more preeclampsia peptides analyzed. However, in contrast to a preeclampsia signature, the weighted levels are defined by a reference dataset, or “training dataset”. Thus, the preeclampsia score is defined by a reference dataset.

These methods of analysis may be readily performed by one of ordinary skill in the art by employing a computer-based system, e.g. using any hardware, software and data storage medium as is known in the art, and employing any algorithms convenient for such analysis. For example, data mining algorithms can be applied through “cloud computing”, smartphone based or client-server based platforms, and the like.

In some instances, the subject methods of determining a preeclampsia peptide representation for a subject further comprise providing the preeclampsia peptide representation as a report. In other words, the subject methods comprise obtaining a biological sample, detecting the abundance of peptide(s) for a preeclampsia peptide panel in the sample, evaluating the detected abundance of peptide in the sample to obtain a preeclampsia peptide representation, and providing, i.e. generating, a report that includes the preeclampsia peptide representation, e.g. preeclampsia peptide profile, preeclampsia peptide signature, or preeclampsia peptide score, etc. Thus, a subject method may further include a step of generating or outputting a report providing the results of an evaluation the abundance of preeclampsia peptide(s) in a biological sample, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

The preeclampsia peptide representation that is so obtained may then be employed in the clinic, e.g. in methods for diagnosing, prognosing, or treating preeclampsia. For example, the marker level representation may be employed to predict if a pregnant woman will develop preeclampsia, to diagnose preeclampsia in a pregnant woman, to characterize a diagnosed preeclampsia, to determine a therapy for preeclampsia, to monitor the responsiveness of the pregnant to treatment for preeclampsia, etc. as described herein. In other words, a medical practitioner will be able to provide a diagnosis, prognosis, or treatment for preeclampsia or monitor a preeclampsia based upon the obtained preeclampsia peptide representation. In some instances, the measurement of particular combinations of preeclampsia markers disclosed herein provides for a preeclampsia prognosis that has an improved accuracy over a preeclampsia prognosis made using standard methods known in the art.

In some embodiments, the preeclampsia peptide representation is employed by comparing it to a reference, to identify similarities or differences with the reference, where the similarities or differences that are identified are then employed to predict if a pregnant woman will develop preeclampsia, to diagnose preeclampsia in a pregnant woman, to characterize a diagnosed preeclampsia, to monitor the responsiveness of the pregnant to treatment for preeclampsia, etc. For example, a reference may be a sample from an individual that has preeclampsia (i.e. a positive control) or that does not have preeclampsia (i.e. a negative reference), which may be used, for example, as a reference/control in the evaluation of the preeclampsia peptide representation for a given patient. As another example, a reference may be a preeclampsia peptide representation, e.g. profile, signature or score, which is representative of a preeclampsia state, i.e. as determined by the analysis of one or more individuals having preeclampsia (i.e. a positive reference), or a preeclampsia peptide representation, e.g. profile, signature or score, which is representative of a healthy individual, i.e. as determined by the analysis of one or more healthy individuals (i.e. a negative reference), and may be used as a reference/control to interpret the marker level representation of a given subject. As indicated above, the reference may be a positive reference/control, e.g., a sample or peptide representation thereof from a pregnant woman that has preeclampsia, or that will develop preeclampsia, or that has preeclampsia that is manageable by known treatments, or that has preeclampsia that has been determined to be responsive only to the delivery of the baby. Alternatively, the reference may be a negative reference/control, e.g. a sample or peptide representation thereof from a pregnant woman that has not developed preeclampsia, or a woman that is not pregnant. References are preferably the same type of sample or, if peptide representations, are obtained from the same type of sample as the sample that was employed to generate the peptide representation for the individual being monitored. For example, if the serum of an individual is being evaluated, the reference/control would preferably be of serum.

In certain embodiments, the obtained peptide representation is compared to a reference to obtain information regarding the individual being tested for preeclampsia. In certain embodiments, the obtained peptide representation is compared to two or more references. For example, the obtained marker level representation may be compared to a negative reference and a positive reference to obtain confirmed information regarding if the individual will develop preeclampsia. As another example, the obtained marker level representation may be compared to a reference that is representative of a preeclampsia that is responsive to treatment and a reference that is representative of a preeclampsia that is not responsive to treatment to obtain information as to whether or not the patient will be responsive to treatment.

The comparison of the obtained preeclampsia peptide representation and the one or more references may be performed using any convenient methodology, where a variety of methodologies are known to those of skill in the art. For example, those of skill in the art of arrays will know that array profiles may be compared by, e.g., comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing marker level profiles are also described above. Similarly, those of skill in the art of ELISAs will know that ELISA data may be compared by, e.g. normalizing to standard curves, comparing normalized values, etc. The comparison step results in information regarding how similar or dissimilar the obtained peptide representation is to the control/reference, which similarity/dissimilarity information is employed to prognose preeclampsia, for example to predict the onset of a preeclampsia, diagnose preeclampsia, monitor a preeclampsia patient, etc. Similarity may be based on relative peptide abundance, absolute peptide abundance or a combination of both. In certain embodiments, a similarity determination is made using a computer having a program stored thereon that is designed to receive input for a peptide representation obtained from a subject, e.g., from a user, determine similarity to one or more reference peptide representations, and return a preeclampsia prognosis, e.g., to a user (e.g., lab technician, physician, pregnant individual, etc.).

Depending on the type and nature of the reference/control profile(s) to which the obtained preeclampsia peptide representation is compared, the above comparison step yields a variety of different types of information regarding the cell/bodily fluid that is assayed. As such, the above comparison step can yield a positive/negative prediction of the onset of preeclampsia. Alternatively, such a comparison step can yield a positive/negative diagnosis of preeclampsia. Alternatively, such a comparison step can provide a characterization of a preeclampsia.

In other embodiments, the preeclampsia peptide representation is employed directly, i.e. without comparison to a reference, to make a prediction, diagnosis, or characterization.

In some embodiments, other analyses may be employed in conjunction with the aforementioned preeclampsia peptide representation to provide a preeclampsia prognosis/diagnosis for the individual. Such analyses are well known in the art, and include, for example, an analysis of polypeptide and peptide markers known in the art to be associated with preeclampsia, e.g. VEGF-R1 (also known as sFlt-1; Genbank Accession Nos. NM_{—}001159920.1 (isoform 2), NM_{—}001160030.1 (isoform 3), and NM_{—}001160031.1 (isoform 4)) (Verlohren et al. (2010) Amer Journal of Obstetrics and Gynecology 161: e1-e11); PIGF (Genbank Accession Nos.NM_{—}002632.5 (isoform 1) and NM_{—}001207012.1 (isoform 2)) (Verlohren et al., supra); and preeclampsia markers described in, e.g., US Publication No. 2010/0297679, US Publication No. 2010/0163721, US Publication No. 2012/0149041, and U.S. Provisional Application No. 61/731,640, the full disclosures of which are incorporated herein by reference.

In some instances, the method further comprises detecting one or more clinical parameter, and providing a prognosis based on the level of biomarker peptides and these one or more clinical parameters. Preeclampsia is a multisystem complication of pregnancy characterized by high blood pressure, e.g. 140/90 mm/Hg or higher, and protein in the urine (proteinuria). Other symptoms of preeclampsia include swelling of the hands and face/eyes (edema), sudden weight gain over 1-2 days or more than 2 pounds a week, higher-than-normal liver enzymes, and a platelet count of less than 100,000 (thrombocytopenia). Preeclampsia typically occurs in the third trimester of pregnancy, but in severe cases, the disorder occurs in the 2d trimester, after about the 22^{nd} week of pregnancy. Thus, in some instances, the method further comprises measuring one or more clinical parameters selected from blood pressure, protein in urine, water retention, weight, liver enzymes, and platelet count, where high blood pressure (e.g. 140/90 mm/Hg or higher), proteinuria, edema, sudden weight gain over 1-2 days or more than 2 pounds a week, higher-than-normal liver enzymes, or a platelet count of less than 100,000 (thrombocytopenia) in combination with a preeclampsia score that is comparable to a preeclampsia reference is indicative of preeclampsia.

In some embodiments, the subject methods of prognosing or diagnosing preeclampsia include providing a prediction, diagnosis, or characterization of preeclampsia. In such embodiments, the prediction, diagnosis, or characterization may be provided by providing, i.e. generating, a written report that includes the practitioner's monitoring assessment, i.e. the practitioner's prediction of the onset of preeclampsia (a “preeclampsia prediction”), the practitioner's diagnosis of the subject's preeclampsia (a “preeclampsia diagnosis”), or the practitioner's characterization of the subject's preeclampsia (a “preeclampsia characterization”). Thus, a subject method may further include a step of generating or outputting a report providing the results of a monitoring assessment, which report can be provided in the form of an electronic medium (e.g., an electronic display on a computer monitor), or in the form of a tangible medium (e.g., a report printed on paper or other tangible medium). Any form of report may be provided, e.g. as known in the art or as described in greater detail below.

### Reports

A “report,” as described herein, is an electronic or tangible document which includes report elements that provide information of interest relating to a subject monitoring assessment and its results. In some embodiments, a subject report includes at least a preeclampsia peptide representation, e.g. as an aspect of the subject methods directed to obtaining a preeclampsia peptide representation, discussed in greater detail above. In some embodiments, a subject report includes at least a preeclampsia prediction, preeclampsia diagnosis, or preeclampsia characterization, i.e. a prediction as to the likelihood of a patient developing preeclampsia, a diagnosis of preeclampsia, or a characterization of the preeclampsia, respectively, e.g. as an aspect of the subject methods directed to providing a preeclampsia prognosis or diagnosis for an individual, discussed in greater detail above. A subject report can be completely or partially electronically generated. A subject report can further include one or more of: 1) information regarding the testing facility; 2) service provider information; 3) patient data; 4) sample data; 5) an assessment report, which can include various information including: a) reference values employed, and b) test data, where test data can include, e.g., a preeclampsia peptide representation; 6) other features.

The report may include information about the testing facility, which information is relevant to the hospital, clinic, or laboratory in which sample gathering and/or data generation was conducted. Sample gathering can include obtaining a fluid sample, e.g. blood, saliva, urine etc.; a tissue sample, e.g. a tissue biopsy, etc. from a subject. Data generation can include measurements of the abundance of preeclampsia peptides. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a patient data section, including patient medical history (which can include, e.g., age, race, serotype, prior preeclampsia episodes, and any other characteristics of the pregnancy), as well as administrative patient data such as information to identify the patient (e.g., name, patient date of birth (DOB), gender, mailing and/or residence address, medical record number (MRN), room and/or bed number in a healthcare facility), insurance information, and the like), the name of the patient's physician or other health professional who ordered the monitoring assessment and, if different from the ordering physician, the name of a staff physician who is responsible for the patient's care (e.g., primary care physician).

The report may include a sample data section, which may provide information about the biological sample analyzed in the monitoring assessment, such as the source of biological sample obtained from the patient (e.g. blood, saliva, or type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include values associated with one or more reference samples. The interpretive report can include a prediction of the likelihood that the subject will develop preeclampsia. The interpretive report can include a diagnosis of preeclampsia. The interpretive report can include a characterization of preeclampsia. The interpretive report can include, for example, the results of a peptide detection assay (e.g., “1.5 nmol/liter EDPQGDAAQKTDT in serum”); an evaluation of the results of the peptide detection assay (e.g. “a preeclampsia peptide score of 0.2”) and interpretation, i.e. prediction, diagnosis, or characterization. The assessment portion of the report can optionally also include a recommendation(s). For example, where the results indicate that preeclampsia is likely, the recommendation can include a recommendation that diet be altered, blood pressure medicines administered, etc., as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g. prediction, diagnosis or characterization of preeclampsia).

### Utility

Methods and compositions of the present disclosure find use in prognosing, diagnosing, and/or treating preeclampsia. By “prognosing” and “providing a prognosis” it is generally meant providing a prediction of a subject's susceptibility to a disease or disorder, i.e. preeclampsia; providing a determination, or diagnosis, as to whether a subject is presently affected by a disease or disorder, i.e. preeclampsia; providing a prediction for a subject affected by a disease or disorder (e.g., determination of the severity of preeclampsia, likelihood that a preeclampsia condition will develop into eclampsia); providing a prediction of a subject's responsiveness to treatment for the disease or disorder; and monitoring a subject's condition to provide information as to the effect or efficacy of therapy. In other words, the subject methods and compositions may be used to make a prediction of a subject's susceptibility to a disease or disorder, i.e. preeclampsia; make a determination, or diagnosis, as to whether a subject is presently affected by a disease or disorder, i.e. preeclampsia; make a prediction for a subject affected by a disease or disorder (e.g., determination of the severity of preeclampsia, likelihood that a preeclampsia condition will develop into eclampsia); make a prediction of a subject's responsiveness to treatment for the disease or disorder; and monitor a subject's condition to provide information as to the effect or efficacy of therapy. By “predicting if the individual will develop preeclampsia”, it is meant determining the likelihood that an individual will develop preeclampsia in the next week, in the next 3 weeks, in the next 5 weeks, in the next 2 months, in the next 3 months, e.g. during the remainder of the pregnancy. By “diagnosing preeclampsia,” it is meant determining that the individual has developed preeclampsia, i.e. a hypertension due to the pregnancy, or pregnancy-induced hypertension. By “characterizing a preeclampsia” it is meant determining the extent of preeclampsia in the individual, e.g. to monitor the individual, determine therapeutic regimen, etc. as is well known in the art. The terms “individual,” “subject,” “host,” and “patient,” are used interchangeably herein and refer to any mammalian subject for whom diagnosis, treatment, or therapy is desired, particularly humans.

In some aspects, the subject methods find use in treating an individual for preeclampsia. By “treatment”, “treating” and the like it is generally meant obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. The subject therapy may be administered prior to the symptomatic stage of the disease, and in some cases after the symptomatic stage of the disease. For example, the disclosed methods may be used to diagnose or prognose an individual having preeclampsia or at risk for having preeclampsia, and a treatment regimen provided based said diagnosis/prognosis. In some such instances, the method further comprises prescribing a preeclampsia treatment. In some such instances, the treatment is bed rest, drinking extra water, a low salt diet, medicines to control blood pressure, or corticosteroids.

In some instances, the measurement of the preeclampsia peptide panels disclosed herein provides for a preeclampsia prognosis that has an improved specificity, sensitivity, and accuracy over a preeclampsia prognosis or diagnosis made using standard methods known in the art. By sensitivity, also called the “recall rate” in some fields, it is meant the proportion of actual positives which are correctly identified as such (e.g. the percentage of individuals at risk for developing preeclampsia that really are at risk for developing preeclampsia). By specificity, it is meant the proportion of actual negatives which are correctly identified as such (e.g. the percentage of healthy people that are correctly identified as not being at risk for developing preeclampsia). By accuracy, it is meant the degree of closeness of measurements of a quantity to that quantity's true value (e.g. the percentage of true results overall that are correctly called, i.e. the percentage of individuals at risk for developing preeclampsia that accurately identified plus the percentage of healthy individuals that accurately identified). Mathematically, these terms may be defined as follows:

$$Sensitivity = \frac{( Number of true positives )}{( \begin{matrix}Number of true positives + \\ Number of false negatives\end{matrix} )}$$

$$Specificity = \frac{( Number of true negatives )}{( \begin{matrix}Number of true negatives + \\ Number of false positives\end{matrix} )}$$

$$Accuracy = \frac{( Number of true positives + true negatives )}{( \begin{matrix}Number of true positives + false positives + \\ false negatives + true negatives\end{matrix} )}$$

For example, the 19-peptide preeclampsia panel provided in Table 4 provides a sensitivity of 100%, a specificity of 80% or better, and an accuracy of 90%. The sensitivity, specificity and accuracy of other preeclampsia peptide panels encompassed herein may be readily determined using the above mathematical formulas.

### Reagents, Devices and Kits

Also provided are reagents, devices and kits thereof for practicing one or more of the above-described methods. The subject reagents, systems and kits thereof may vary greatly. Reagents of interest include reagents specifically designed for use in producing the above-described preeclampsia peptide representations from a sample, for example, one or more detection elements, e.g. antibodies or mass spec reagents for the detection of peptide. In some instances, the detection element comprises reagent(s) to detect one or more peptide markers, for example, the detection element may be a dipstick, a plate, an array, or cocktail that comprises one or more detection elements, e.g. one or more antibodies, which may be used to detect the expression of one or more preeclampsia peptide markers simultaneously,

One type of reagent that is specifically tailored for generating peptide representations, e.g. preeclampsia peptide representations, is a collection of isotope labeled- and unlabeled-peptides that may be used for calibration and as internal references, e.g. in spectrometry methods, e.g. mass spectrometry (MS)-based methods.

Another type of reagent that is specifically tailored for generating peptide representations, e.g. preeclampsia peptide representations, is a collection of antibodies that bind specifically to the preeclampsia peptides of interest, e.g. in an ELISA format, in an xMAP™ microsphere format, on a proteomic array, in suspension for analysis by flow cytometry, by western blotting, by dot blotting, or by immunohistochemistry. Usually, the antibodies are specific for the preeclampsia peptide marker(s) of interest but not the polypeptide(s) from which they were derived. Typically, such antibodies will be specific for a domain created by the cleavage event that generated the peptide. Antibodies that are specific to the polypeptide(s) and not the peptide marker(s) may also be included, which serve as negative control(s).

In some instances, a system may be provided. As used herein, the term “system” refers to a collection of reagents, however compiled, e.g., by purchasing the collection of reagents from the same or different sources. In some instances, a kit may be provided. As used herein, the term “kit” refers to a collection of reagents provided, e.g., sold, together. For example, the peptide-based detection of the sample may be coupled with data processing platform that will allow multiparameter determination of the subject peptide biomarkers for personalized preeclampsia care.

The systems and kits of the subject invention may include the above-described peptides or peptide-specific antibody collections. The systems and kits may further include one or more additional reagents employed in the various methods, such as liquid chromatography columns, e.g. HPLC columns, for initial purification of the peptides, fractionation vials, etc., various buffer mediums, e.g. hybridization and washing buffers, labeled probe purification reagents and components, like spin columns, etc., signal generation and detection reagents, e.g. labeled secondary antibodies, streptavidin-alkaline phosphatase conjugate, chemifluorescent or chemiluminescent substrate, and the like.

The subject systems and kits may also include a reference, which element is, in many embodiments, a control sample or control biomarker representation that can be employed, e.g., by a suitable experimental or computing means, to make a preeclampsia prognosis based on an “input” marker level profile, e.g., that has been determined with the above described reference. Representative references include samples from an individual known to have or not have preeclampsia, databases of preeclampsia peptide representations, e.g., reference or control signatures or scores, and the like, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

### Background

Preeclampsia (PE) complicates about 5% of all pregnancies worldwide and is a major cause of maternal, fetal and neonatal morbidity and mortality, especially in developing nations (Venkatesha S, et al. (2006) Soluble endoglin contributes to the pathogenesis of preeclampsia. Nat Med 12: 642-649; Levine R J, et al. (2006) Soluble endoglin and other circulating antiangiogenic factors in preeclampsia. N Engl J Med 355: 992-1005). It is a potentially dangerous complication of the second half of pregnancy, labor, or early period after delivery, characterized by hypertension, abnormal amounts of protein in the urine, and other systemic disturbances. PE currently has little effective therapy, though it largely resolves after placenta and fetus delivery (Powe C E, et al. (2011) Preeclampsia, a disease of the maternal endothelium: the role of antiangiogenic factors and implications for later cardiovascular disease. Circulation 123: 2856-2869). PE is one of the most common reasons for induced preterm delivery (Redman C W, Sargent I L (2005) Latest advances in understanding preeclampsia. Science 308: 1592-1594).

The use of biofluid (e.g. serum or urine) for the analysis of the naturally occurring peptidome (MW<4000) as a source of biomarkers has been reported in different diseases (Ling X B, et al. (2010) Urine Peptidomic and Targeted Plasma Protein Analyses in the Diagnosis and Monitoring of Systemic Juvenile Idiopathic Arthritis. Clin Proteomics 6: 175-193; Ling X B, et al. (2011) A diagnostic algorithm combining clinical and molecular data distinguishes Kawasaki disease from other febrile illnesses. BMC Med 9: 130; Ling X B, et al. (2010) Urine peptidomics for clinical biomarker discovery. Advances in clinical chemistry 51: 181-213; Ling X B, et al. (2010) Integrative urinary peptidomics in renal transplantation identifies biomarkers for acute rejection. J Am Soc Nephrol 21: 646-653; Villanueva J, et al. (2006) Differential exoprotease activities confer tumor-specific serum peptidome patterns. J Clin Invest 116: 271-284). For clinical application, mass spectrometry-based profiling of naturally occurring peptides can provide an extensive inventory of serum peptides derived from either high-abundant endogenous circulating proteins or cell and tissue proteins (Liotta L A, Petricoin E F (2006) Serum peptidome for cancer detection: spinning biologic trash into diagnostic gold. J Clin Invest 116: 26-30). These peptides are usually soluble, and stable from endogenous proteases or peptidases, and can be directly used for liquid chromatography-mass spectrometry (LC/MS) analysis without additional manipulation (e.g. tryptic digests). However, a serum peptidomics based approach has not been attempted for the discovery of PE biomarkers.

We hypothesized that there would be differential serum peptidomic signatures reflective of a PE-specific alteration of proteolytic and anti-proteolytic pathways. Our peptidomics-based discovery and subsequent validation yielded 19 unique serum peptides differing between PE and control subjects. These peptide biomarkers, collectively as a panel, can effectively assess PE.

### Materials and Methods

Specimen Collection and Preprocessing.

To identify the PE related peptide sequences, case and control cohorts were constructed to match gestational age, ethnicity, and parity. Serum specimens from 62 pregnant women (PE n=31, control n=31) were purchased from ProMedDX Inc. (Norton, Mass. 02766, http://www.promeddx.com). The PE patients were diagnosed with preeclampsia characterized by both hypertension and proteinuria. As shown in Table 1, all of the 31 PE patients had both hypertension and proteinuria; 41.9% of them had headache; 22.6% of them had edema; and 25.8% of them had other additional symptoms. The 62 samples were divided into two datasets randomly: the training set (n=21 case group, n=21 control group); the testing set (n=10 case group, n=10 control group). The demographics on the 2 sets (training and testing) were summarized in Table 2, which compares the ethnicity, age and gestation delivery time of the case and control samples (continuous variable: two-tailed Mann-Whitney U test; categorical analysis: Fisher's exact test).

TABLE 1
PE patients' presenting signs and symptoms.

| Presenting Signs and | | |
|---|---|---|
| Symptoms | Number (percentage) | |
| Hypertension | 31 | (100%) |
| Proteinuria | 31 | (100%) |
| Headache | 13 | (41.9%) |
| Edema | 7 | (22.6%) |
| Others | 8 | (25.8%) |

TABLE 2
Demographics.

| | Training data | | | Testing data | | | |
|---|---|---|---|---|---|---|---|
| | PE | control | | PE | control | | Overall |
| Characteristic | n = 21 (50%) | n = 21 (50%) | p value | n = 10 (50%) | n = 10 (50%) | p value | p value |
| Ethnicity | | | 0.512 | | | 0.164 | 0.286 |
| African American | 6 (28.6%) | 5 (23.8%) | | 1 (10%) | 4 (40%) | | |
| Asian | 2 (9.5%) | 0 (0%) | | 0 (0%) | 0 (0%) | | |
| Hispanic | 11 (52.4%) | 15 (71.4%) | | 7 (70%) | 6 (60%) | | |
| Other | 2 (9.5%) | 1 (4.8%) | | 2 (20%) | 0 (0%) | | |
| Age (year) | | | | | | | |
| median (IQR) | 24 (19.32) | 24 (20.29) | 0.95 | 23 (20.32) | 24 (19.26) | 1 | 0.916 |
| Week of gestation | | | | | | | |
| median (IQR) | 36 (33.37) | 33 (28.36) | 0.077 | 33.5 (28.37) | 37.5 (35.38) | 0.087 | 0.772 |

Serum peptides were prepared as previously described in Ling X B, et al. (2010) Urine peptidomics for clinical biomarker discovery. Advances in clinical chemistry 51: 181-213. Serum samples were processed by centrifugal filtration at 3000×g for 20 min at 10° C. through Amicon Ultra centrifugal filtration devices (10 kDa cutoff) (Millipore, Bedford, Mass.) preequilibrated with 10 ml Milli-Q water. The filtrate (serum peptidome) containing the low MW naturally occurring peptides was processed with Waters Oasis HLB Extraction Cartridges (Waters Corporation, Milford, Mass.), and extracted with ethyl acetate. The serum peptide samples were quantified by the 2,4,6-trinitrobenzenesulfonic acid (TNBS) assay, as described in (Snyder S L, Sobocinski P Z (1975) An improved 2,4,6-trinitrobenzenesulfonic acid method for the determination of amines. Anal Biochem 64: 284-288). Lyophilized human serum peptide samples were reconstituted in 2% acetonitrile with 0.1% formic acid and separated on a Paradigm MS4 liquid chromatography system (Michrom BioResources, Auburn, Calif.) with a 60 min linear gradient of 5-95% buffer A to B (buffer A: 2% acetonitrile with 0.1% formic acid in H₂O, buffer B: 90% acetonitrile with 0.1% formic acid in H₂O) at a flow rate of 2 μl/min using a 0.2×50 mm 3μ 200A Magic C18AQ column (Michrom BioResources, Auburn, Calif.). Each randomized sample run was followed by a 60 min wash run. The fractionated peptides were directly applied to an LTQ ion trap mass spectrometer (Thermo Fisher Scientific, San Jose, Calif.) equipped with a Fortis tip mounted nano-electrospray ion source (AMR, Tokyo, Japan). The Fortis tip is with 150 μm outside diameter (OD) and 20 μm inside diameter (ID), which can be used with flow rates between 200-2000 nl/min. The electrospray voltage was set at 1.8 kV. Each full MS scan with a mass range of 400-2000 m/z was followed by two data-dependent scans of the two most abundant ions observed in the first full MS scan. MS/MS spectra were generated for the highest peak in each scan with the relative collision energy for MS/MS set to 35%. Raw MS/MS data were preprocessed, as previously described (Griffin N M, et al. (2010) Label-free, normalized quantification of complex mass spectrometry data for proteomic analysis. Nat Biotechnol 28: 83-89), before further statistical analysis. Peptide protein identification was search against the human SwissProt database as previously described. At first, the intensity values of the same peptides in the same proteins were summed up across different fractions for each sample. Therefore, each peptide in one sample has one intensity value, which was later normalized by the total intensity value of all peptides found in the sample.

Feature Selection to Identify Discriminative PE Serum Peptide Biomarkers.

612 peptides, across all samples, were identified by MS and MS/MS steps and chosen as the biomarker candidates. Significance analysis of microarrays (SAM (Tusher V G, et al. (2001) Significance analysis of microarrays applied to the ionizing radiation response. Proc Natl Acad Sci USA 98: 5116-5121)) was used to calculate d-scores indicating the relative positive (increased) and negative (decreased) changes in abundance of these serum peptides in PE subjects in comparison to control subjects. SAM calculated a minimal false discovery rate (q value) for significance.

A shrunken centroid algorithm called predictive analysis of microarrays (PAM (Tibshirani R, et al. (2002) Diagnosis of multiple cancer types by shrunken centroids of gene expression. Proc Natl Acad Sci USA 99: 6567-6572)) was used to find and construct a PE-specific serum peptide panel. 42 samples, balanced in PE and control samples, were randomly selected as the training data of PAM, and the rest 20 samples were used as the testing data. With the training data, training and 100 repeated random sub-sampling cross validation was used to train the PAM model, select the significant features for the diagnostic panel and estimate the prediction error. A threshold was used in the PAM algorithm to control the number of shrunken centroids. A larger threshold will result in a smaller number of shrunken centroids. Generally, as the number of shrunken centroids, namely, selected biomarkers, increases, the prediction error of both the training samples and testing samples will decrease. The estimated PE score of each sample was computed based on the predicted probability of the PAM model (19-peptide panel). In PAM algorithm, a sample was predicted as a PE sample if the score was larger than 0.5. The predictive performance of each biomarker panel analysis was evaluated by sensitivity and specificity analysis.

ELISA Assays Validating PE Marker Candidates.

ELISA assays were performed using commercial kits following vendors' instructions. All assays were performed to measure serum levels of placental growth factor (PIGF), R&D system Inc. (MN, US) and soluble fms-like tyrosine kinase (sFlt-1), R&D system Inc.

### Results

Sample Qualification with sFlt-1 and PIGF Analysis.

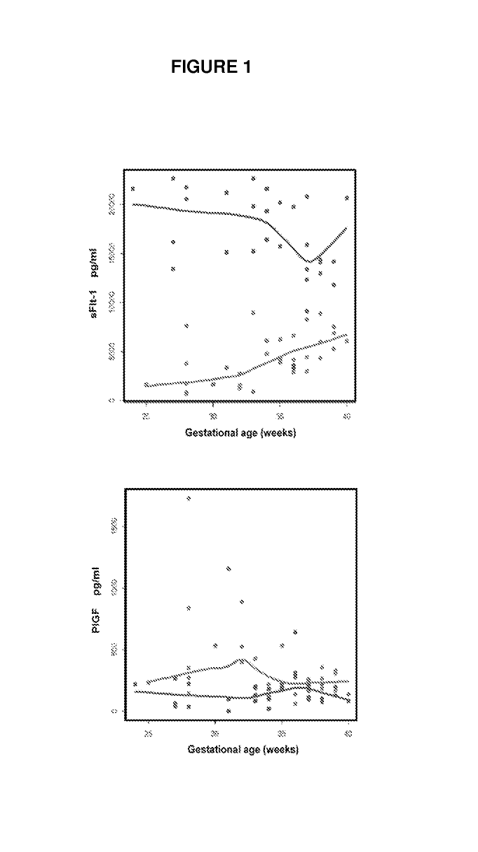

Elevated soluble sFlt-1 and decreased PIGF levels are suggested in the pathogenesis of PE (Shibata E, et al. (2005) Soluble fms-like tyrosine kinase 1 is increased in preeclampsia but not in normotensive pregnancies with small-for-gestational-age neonates: relationship to circulating placental growth factor. J Clin Endocrinol Metab 90: 4895-4903; Maynard S E, et al. (2003) Excess placental soluble fms-like tyrosine kinase 1 (sFlt1) may contribute to endothelial dysfunction, hypertension, and proteinuria in preeclampsia. J Clin Invest 111: 649-658; Wolf M, et al. (2005) Circulating levels of the antiangiogenic marker sFLT-1 are increased in first versus second pregnancies. Am J Obstet Gynecol 193: 16-22; Rajakumar A, et al. (2005) Extra-placental expression of vascular endothelial growth factor receptor-1, (Flt-1) and soluble Flt-1 (sFlt-1), by peripheral blood mononuclear cells (PBMCs) in normotensive and preeclamptic pregnant women. Placenta 26: 563-573; Taylor A P, et al. (2003) Altered tumor vessel maturation and proliferation in placenta growth factor-producing tumors: potential relationship to post-therapy tumor angiogenesis and recurrence. Int J Cancer 105: 158-164; Tidwell S C, et al. (2001) Low maternal serum levels of placenta growth factor as an antecedent of clinical preeclampsia. Am J Obstet Gynecol 184: 1267-1272; Torry D S, et al. (1998) Preeclampsia is associated with reduced serum levels of placenta growth factor. Am J Obstet Gynecol 179: 1539-1544), and the sFlt-1/PIGF ratio has been proposed as a useful index in the diagnosis and management of PE (Verlohren S, et al. (2010) An automated method for the determination of the sFlt-1/PIGF ratio in the assessment of preeclampsia. Am J Obstet Gynecol 202: 161 e161-161 e111; Esplin M S, et al. (2011) Proteomic identification of serum peptides predicting subsequent spontaneous preterm birth. Am J Obstet Gynecol 204: 391 e391-398). Our ELISA assay result (FIG. 1) reproduced previous observations (Verlohren et al (2010), supra: Esplin et al. (2011) supra). With the range of gestation-week 24 to 40, the control PIGF serum concentrations increased continuously peaked around gestation week 30 and then decreased to the end of the pregnancy. The control sFlt-1 serum concentrations remained relatively stable trending slightly upwards with the gestation weeks. When comparing PE to control subjects, these two analytes' serum concentrations were differentiated with sFlt-1 significantly increased and PIGF significantly decreased throughout the gestation weeks. Our ELISA analysis results provided a sample qualification analysis indicating that our PE and control samples can be used to allow further biomarker discovery and testing analyses.

PE Peptide Biomarker Identification.

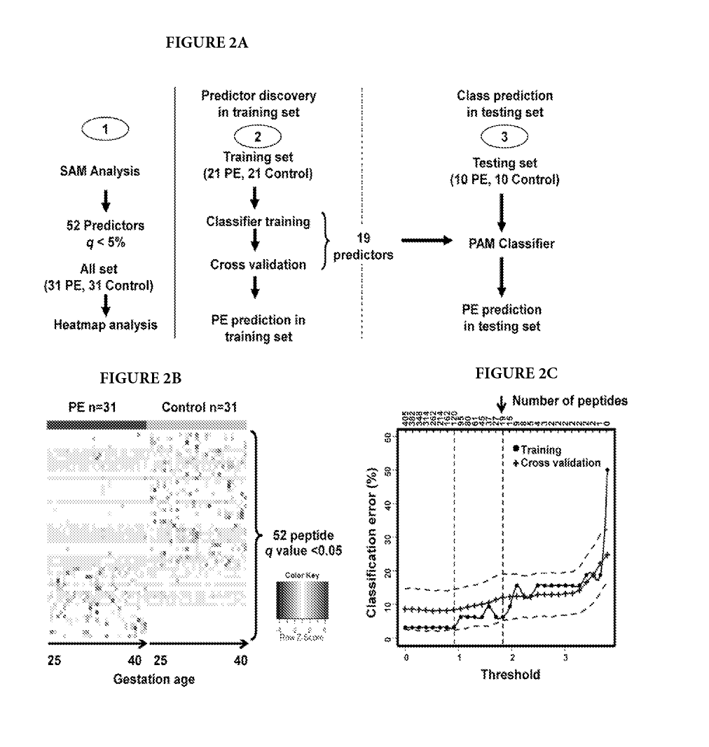

FIG. 2A diagrams the PE discriminant peptide biomarker selection, predictive panel construction and validation processes. Initial statistical analysis of the training set by SAM (Tusher V G, et al. (2001) Significance analysis of microarrays applied to the ionizing radiation response. Proc Natl Acad Sci USA 98: 5116-5121) algorithm identified 52 peptides derived from 14 protein precursors with highly significant differences in expression (q<5%) between PE and control samples (Table 3). Consistent with the significance findings, heat map plotting (FIG. 2B) demonstrated that a differential pattern of the 52 peptides collectively arranged all the samples according to PE and control groups. These results show that the serum abundances of peptide biomarkers are differential between PE and control subjects. In addition, when the heatmap data were sorted according to the gestational age for both PE and control groups, no obvious differential pattern was observed between early and late gestation.

TABLE 3
Serum peptides identified by SAM algorithm (q value < 0.05), which are
significantly differentiated between PE and control subjects.

| Heatmap | | | | |
|---|---|---|---|---|
| index | Protein | Peptide sequence | Score(d) | q-value(%) |
| 1 | FGA | (R)GSESGIFTNTKE(S) | 6.141762 | 0 |
| 2 | FGA | (G)SEADHEGTHST(K) | 5.186152 | 0 |
| 3 | KNG1 | (K)LDDDLEHQ(G) | 3.857129 | 0 |
| 4 | TMSB4 | (P)SKETIEQEKQAGES(-) | 3.688479 | 0 |
| 5 | FGA | (G)SESGIFTNTKE(S) | 3.622314 | 0 |
| 6 | C3 | (R)SEETKENEGFTV(T) | 3.536669 | 0 |
| 7 | TMSB4 | (S)KETIEQEKQAGES(-) | 3.478967 | 0 |
| 8 | APO-A4 | (G)NTEGLQ(K) | 3.369214 | 0 |
| 9 | FGA | (A)DEAGSEADHEGTH(S) | 3.364307 | 0 |
| 10 | FGA | (E)GDFLAEGGGV(R) | 3.255781 | 0 |
| 11 | FGA | (A)DEAGSEADHEGT(H) | 3.157053 | 0 |
| 12 | FGA | (R)GSESGIFTNTKESS(S) | 3.10426 | 0 |
| 13 | FGA | (A)DEAGSEADHEGTHST(K) | 2.973072 | 0 |
| 14 | APO-E | (A)TVGSLAG(Q) | 2.874127 | 0 |
| 15 | TMSB4 | (K)ETIEQEKQAGES(-) | 2.643713 | 0 |
| 16 | APO-A4 | (L)GGHLDQQVEEF(R) | 2.6235 | 0 |
| 17 | APO-C3 | (S)SVQESQVAQQA(R) | 2.567146 | 0 |
| 18 | ITIH4 | (R)LLGLPGPPDVPDHAAYHPF(R) | 2.554118 | 0 |
| 19 | APO-L1 | (R)VTEPISAESGEQVER(V) | 2.520311 | 0 |
| 20 | C3 | (R)SEETKENEGF(T) | 2.504033 | 0 |
| 21 | FGA | (G)SESGIFTNTKESS(S) | 2.409848 | 1.893749 |
| 22 | APO-E | (L)DEVKEQVAEV(R) | 2.392038 | 1.893749 |
| 23 | ZYX | (R)GPPASSPAPAPK(F) | 2.34915 | 1.893749 |
| 24 | KNG1 | (R)IGEIKEETT(V) | 2.341402 | 1.893749 |
| 25 | C3 | (R)SEETKENEGFTVTAEGK(G) | 2.305142 | 1.893749 |
| 26 | APO-A1 | (R)LEALKENGGA(R) | 2.304021 | 1.893749 |
| 27 | APO-C3 | (K)TAKDALSSVQES(Q) | 2.296693 | 1.893749 |
| 28 | C3 | (I)HWESASL(L) | 2.235505 | 3.2824982 |
| 29 | APO-A4 | (I)DQNVEELKG(R) | 2.232068 | 3.2824982 |
| 30 | KNG1 | (K)LDDDLEHQGGHVLDHGH(K) | 2.210918 | 3.2824982 |
| 31 | FGA | (A)DEAGSEADHEGTHSTKR(G) | 2.179585 | 3.2824982 |
| 32 | HRNR | (Y)GSGSGWSSSRGPY(E) | 2.132342 | 3.2824982 |
| 33 | C4A | (R)TLEIPGN(S) | 2.119886 | 4.2609351 |
| 34 | APO-E | (A)VGTSAAPVPSDNH(-) | 2.083623 | 4.2609351 |
| 35 | FGA | (Y)NRGDSTFES(K) | −3.7179 | 0 |
| 36 | FGA | (D)FLAEGGGV(R) | −3.39524 | 0 |
| 37 | FGA | (T)SYNRGDSTFES(K) | −3.2551 | 0 |
| 38 | FGA | (Y)NRGDSTFESKS(Y) | −3.2183 | 0 |
| 39 | FGA | (D)STFESKSY(K) | −2.91552 | 0 |
| 40 | SERPINA1 | (A)EDPQGDAAQKTDT(S) | −2.79146 | 0 |
| 41 | FGA | (G)DFLAEGG(G) | −2.74576 | 0 |
| 42 | FGA | (G)EGDFLAEGGGV(R) | −2.73961 | 0 |
| 43 | FGA | (G)EGDFLAEGGG(V) | −2.73489 | 0 |
| 44 | FGA | (K)MADEAGSEADHEGTHST(K) | −2.68688 | 2.3824584 |
| 45 | FGA | (G)DFLAEGGGV(R) | −2.57087 | 2.3824584 |
| 46 | FGA | (G)DSTFESKSY(K) | −2.52742 | 2.3824584 |
| 47 | FGA | (Q)FTSSTSYNRGDSTFES(K) | −2.42019 | 3.2824982 |
| 48 | FGA | (A)DSGEGDFLAEGGGV(R) | −2.41329 | 3.2824982 |
| 49 | FGA | (K)SYKMADEAGSEADHEGTHST(K) | −2.28074 | 4.2609351 |
| 50 | FGA | (G)DFLAEGGGVR(G) | −2.27309 | 4.2609351 |
| 51 | FGA | (S)YKMADEAGSEADHEGTHST(K) | −2.26121 | 4.2609351 |
| 52 | FGA | (G)DFLAEGGG(V) | −2.23299 | 4.2609351 |

PAM algorithm (Tibshirani R, et al. (2002) Diagnosis of multiple cancer types by shrunken centroids of gene expression. Proc Natl Acad Sci USA 99: 6567-6572) was used to find a biomarker panel for PE assessment. When constructing the biomarker panel for prediction, there is a trade-off between a small number of selected biomarkers and small prediction errors. As shown in FIG. 2C, this minimum error solution (peptide n=120) might be of interest. Here, to obtain a more manageable set of candidates, a tolerance level of prediction error of 10% and a number of biomarkers (n=19) were chosen. The selected biomarker panel (Table 4) contains these 19 unique peptides (13 from fibrinogen alpha (FGA), 1 from alpha-1-antitrypsin (A1AT), 1 from apolipoprotein L1 (APO-L1), 1 from inter-alpha-trypsin inhibitor heavy chain H4 (ITIH4), 2 from kininogen-1 (KNG1), and 1 from thymosin beta-4 (TMSB4), totaling 6 protein precursors respectively). All 19 peptide biomarkers have a minimal false discovery rate q value<0.05.

TABLE 4
Serum peptide biomarkers identified to separate PE and control subjects.

| Proteins | Peptide sequences | MW | Score(d) | q value |
|---|---|---|---|---|
| A1AT | (A)EDPQGDAAQKTDT(S) | 1375.06 | −1.786 | <0.05 |
| APO-L1 | (R)VTEPISAESGEQVER(V) | 1630.45 | 1.945 | <0.05 |
| FGA* | (R)GSESGIFTNTKESS(S) | 1443.27 | 1.55 | <0.05 |
| FGA* | (R)GSESGIFTNTKE(S) | 1269.46 | 4.389 | <0.05 |
| FGA* | (G)SESGIFTNTKE(S) | 1212.38 | 2.959 | <0.05 |
| FGA** | (K)SYKMADEAGSEADHEGTHST(K) | 2123.31 | 2.95 | <0.05 |
| FGA** | (A)DEAGSEADHEGTHST(K) | 1543.09 | 2.135 | <0.05 |
| FGA** | (A)DEAGSEADHEGT(H) | 1216.75 | 3.003 | <0.05 |
| FGA** | (G)SEADHEGTHST(K) | 1169.82 | −1.127 | <0.05 |
| FGA*** | (T)ADSGEGDFLAEGGGV(R) | 1379.5 | −2.365 | <0.05 |
| FGA*** | (A)DSGEGDFLAEGGGV(R) | 1309.06 | −2.41 | <0.05 |
| FGA*** | (G)DFLAEGGGV(R) | 863.416 | −1.836 | <0.05 |
| FGA**** | (Y)NRGDSTFESKSY(K) | 1390.73 | −3.366 | <0.05 |
| FGA**** | (Y)NRGDSTFES(K) | 1011.8 | −2.212 | <0.05 |
| FGA**** | (G)DSTFESKSY(K) | 1063.13 | 1.647 | <0.05 |
| ITI H4 | (R)LLGLPGPPDVPDHAAYH PF(R) | 2010.71 | −2.321 | <0.05 |
| KNG-1 | (K)LDDDLEHQ(G) | 984.17 | −2.319 | <0.05 |
| KNG-1 | (R)IGEIKEETT(V) | 1019.3 | −1.172 | <0.05 |
| TMSB4 | (P)SKETIEQEKQAGES(-) | 1564.06 | −2.745 | <0.05 |

FGA: *cluster 1; **cluster 2; ***cluster 3; ****cluster 4.
Score and minimal false discovery rate (q value) were computed using SAM algorithm.

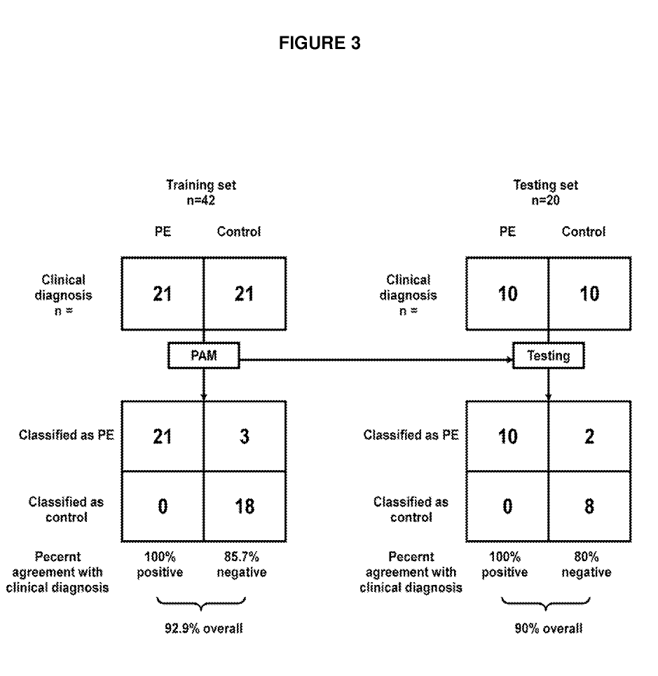

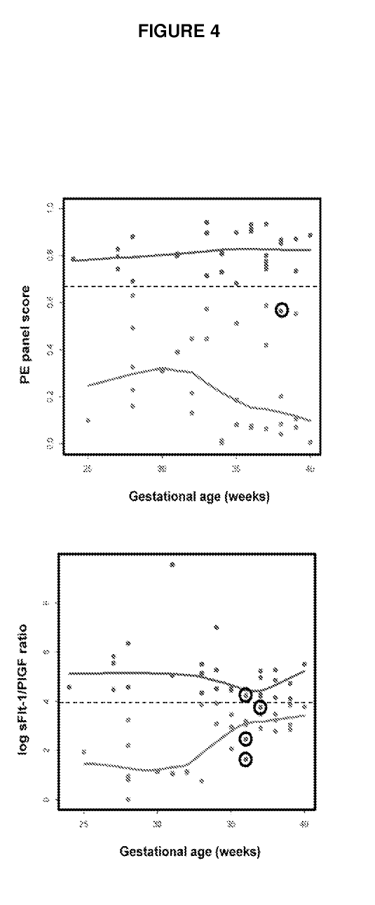

With the selected biomarker panel and trained PAM prediction model, the PE prediction performance was analyzed as in FIG. 3. The left panel of FIG. 3 shows the prediction performance on the training set (n=42), while the right panel of FIG. 3 shows the prediction performance on the blind testing set (n=20). On the training set, all PE samples (n=21) were predicted correctly, while 3 of the 21 (14.3%) control samples were false positive. Thus, the sensitivity on the training set was 85.7% and the specificity was 100%, resulting in the overall prediction accuracy of 92.9%. Similarly, on the testing set, the overall prediction accuracy is 90%, with sensitivity 80% and specificity 100%. The scatter plot of the PAM predicted scores along with gestational ages is shown as in FIG. 4. The predicted score represents the probability of being PE according to the PAM prediction model. Both the prediction accuracy and the scatter plot show that the selected biomarker panel with 19 peptides can be used to effectively predict the occurrence of PE. The early and late gestational age discriminative analyses demonstrated a comparable performance, indicating the potential usefulness of our serum peptide panel in the early diagnosis of PE. The sFlt-1/PIGF ratio's PE assessment utility, previously through the multicenter trial validation (Verlohren S, et al. (2010) An automated method for the determination of the sFlt-1/PIGF ratio in the assessment of preeclampsia. Am J Obstet Gynecol 202: 161 e161-161 e111), was confirmed in this study and used as a benchmark for our newly derived biomarker panels. As shown in FIG. 4, the PE diagnostic performance of our peptide panel was comparable to the sFlt-1/PIGF ratio. If we use 0.66, rather than 0.5, as the cutoff of our PE classification panel, as the dotted line in FIG. 4, there is only 1 misclassified sample. In contrast with it, the sFlt-1/PIGF ratio results to at least 4 misclassified samples.

Pathway Analysis of PE Biomarkers.

We analyzed the 14 parental proteins of the 52-peptide markers (found by SAM with q value<0.05 that are significantly differentially expressed in PE as a composite), using Ingenuity Pathway Analysis software (IPA version 7.6, Ingenuity Systems, Inc., Redwood City, Calif.). Our pathway analysis identified the following statistically significant canonical pathways which may play important roles in the pathophysiology of PE: Liver X receptor (LXR)/retinoid X receptor (RXR) activation (p value 6.31×10⁻¹⁹); atherosclerosis signaling (p value 8.31×10⁻⁴); IL-12 signaling and production in macrophages (p value 9.33×10⁻⁹); clathrin-mediated endocytosis signaling (p value 5.89×10⁻⁹); production of nitric oxide and reactive oxygen species in macrophages (p value 6.17×10⁻⁹); acute phase response signaling (p value 2.24×10⁻⁷); coagulation system (p value 3.09×10⁻⁶); farnesoid X receptor (FXR)/RXR activation (p value 7.24×10⁻⁵); and intrinsic prothrombin activation pathway (p value 2.63×10⁻⁴).

### Discussion

We have employed a serum peptide profiling based approach to identify serum peptide biomarkers that discriminate PE and healthy pregnant controls. 52 significant peptide biomarkers from 14 protein precursors were found, and a 19-peptide biomarker panel was constructed which can diagnose PE with great sensitivity and specificity.

The differential 52 serum peptides are derived from proteins known to be involved in the pathophysiology of PE, e.g. A1AT, APO-L1, FGA, ITIH4, KNG1, SERPINA1 in acute inflammatory and defense response; APO-A4, APO-C3, APO-E, and APO-L1 in lipid metabolism; C3, C4A, FGA, and SERPINA1 in the activation of complement and coagulation responses. This might reflect the nature of PE as a multi-factorial disorder with complicated pathophysiological changes. However, little is known about the function of these peptide fragments.

For both systemic and renal diseases, we previously hypothesized (Ling X B, et al. (2010) Urine peptidomics for clinical biomarker discovery. Advances in clinical chemistry 51: 181-213) that naturally occurring biofluid peptide biomarkers can be the surrogates of pathophysiologies in signaling, proteolytic, and anti-proteolytic pathways. Sequence alignment analyses (Table 4) of these peptides found that FGA peptides line up by forming clusters (n=4) within either the N- or C-terminal end with ladder-like truncations at the opposite ends, suggesting that there is likely disease-specific proteolytic degradation of the parent protein. The peptide biomarkers can be the derivatives of serological proteins, disease specific shedding from other organs, and/or renal-specific proteins, all of which are generated during the proteolysis that occurs in either circulation during systemic diseases or dysfunctional kidneys, and then trimmed down by exoproteases into ladder-like clusters. The discovery of the serum peptide biomarkers for PE supports the notion that PE pathophysiology or pathogenesis can lead to serum specific protein degradation patterns throughout the progression of the disease from early to late gestation. Moreover, our 19-peptide panel predicted well with comparable sensitivity and specificity at either early or late gestational age weeks, indicating its potential utility throughout the disease course and potentially in early onset of PE. This is in contrast to the established use of the sFlt-1/PIGF ratio (Verlohren S, et al. (2010) An automated method for the determination of the sFlt-1/PIGF ratio in the assessment of preeclampsia. Am J Obstet Gynecol 202: 161 e161-161 e111), which works better in early onset but does not have sufficient statistical power to accurately predict late-onset PE.

Interestingly, we have found an ITIH4 peptide (LLGLPGPPDVPDHAAYHPF (SEQ ID NO:47)) as a PE biomarker. This peptide shares an almost identical sequence as a previously published spontaneous preterm birth (SPB) serum peptide biomarker (QLGLPGPPDVPDHAAYHPF (SEQ ID NO:55)) (Esplin M S, et al. (2011) Proteomic identification of serum peptides predicting subsequent spontaneous preterm birth. Am J Obstet Gynecol 204: 391 e391-398) but there is a preceding amino acid sequence change from L to Q (Esplin et al. (2011), supra). Close examination of a database of common gene variations (found on the world wide web at snp.ims.u-tokyo.ac.jp/cgi-bin/SnpInfo.cgi?SNP_ID=IMS-JST073530) revealed that this change is due to the single nucleotide polymorphism (SNP) in ITIH4 where a single coding nucleotide differs from A of amino acid codon cAa to T of cTa, resulting in an amino acid change from Q to L. The exact biological function of ITIH4 and its degraded serum peptide is unknown. Given that the same ITIH4 peptide is a biomarker of both PE and SPB, it is very likely that this is not a disease-process-related biomarker as PE and SPB have very different pathophysiologies.

Serum peptidome biomarker analysis will be useful in diagnosing PE. Technologic advances in multiple reaction monitoring (MRM) (Addona T A, et al. (2009) Multi-site assessment of the precision and reproducibility of multiple reaction monitoring-based measurements of proteins in plasma. Nat Biotechnol 27: 633-641; Anderson L, Hunter C L (2006) Quantitative mass spectrometric multiple reaction monitoring assays for major plasma proteins. Mol Cell Proteomics 5: 573-588), coupled with stable isotope dilution (SID) mass spectrometry (MS) have empowered a “universal” approach to perform quantitative assays for peptides with minimum restrictions, and the ease of assembling multiplex peptide detections in a single measurement. Using common materials and standardized protocols, the reproducibility and transferability of MRM assays between laboratories and across instrument platforms have been demonstrated (Addona T A, et al. (2009) supra). Therefore, in a similar fashion as the current common practice of applying MRM based newborn screening of metabolic diseases, a greater acceptance by the clinical community of SID-MRM-MS technology as a generally applicable approach for biofluid protein and peptide quantification is expected. Detecting our serum peptide PE biomarker panel by using, for example, SID-MRM-MS, will lead to a quick and reliable multiplexed test which can be run routinely in the hospital setting for PE care.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method for diagnosing or prognosing preeclampsia in a subject, the method comprising:
obtaining a preeclampsia peptide representation for a panel of preeclampsia peptides in a blood sample from the subject, and
providing a preeclampsia diagnosis or prognosis based on the preeclampsia peptide representation.

2. The method according to claim 1, wherein the panel comprises 5 or more peptides derived from polypeptides selected from the group consisting of alpha-1-antitrypsin (A1AT), apolipoprotein A-I (APO-A1), apolipoprotein A-IV (APO-A4), apolipoprotein C-III (APO-C3), apolipoprotein E (APO-E), apolipoprotein L 1 (APO-L1), complement component 3 (C3), complement component 4A (C4A), fibrinogen alpha chain (FGA), hornerin (HRNR), inter-alpha-trypsin inhibitor heavy chain H4 (ITIH4), kininogen-1 (KNG-1), thymosin beta-4-like protein 1 (TMSB4), and zyxin (ZYX).

3. The method according to claim 2, wherein the panel comprise 5 or more peptides selected from the group consisting of EDPQGDAAQKTDT (SEQ ID NO:1), LEALKENGGA (SEQ ID NO:2), NTEGLQ (SEQ ID NO:3), GGHLDQQVEEF (SEQ ID NO:4), DQNVEELKG (SEQ ID NO:5), SVQESQVAQQA (SEQ ID NO:6), TAKDALSSVQES (SEQ ID NO:7), TVGSLAG (SEQ ID NO:8), DEVKEQVAEV (SEQ ID NO:9), VGTSAAPVPSDNH (SEQ ID NO:10), VTEPISAESGEQVER (SEQ ID NO:11), SEETKENEGFTV (SEQ ID NO:12), SEETKENEGF (SEQ ID NO:13), SEETKENEGFTVTAEGK (SEQ ID NO:14), HWESASL (SEQ ID NO:15), TLEIPGN (SEQ ID NO:16), GSESGIFTNTKE (SEQ ID NO:17), SEADHEGTHST (SEQ ID NO:18), SESGIFTNTKE (SEQ ID NO:19), DEAGSEADHEGTH (SEQ ID NO:20), GDFLAEGGGV (SEQ ID NO:21), DEAGSEADHEGT (SEQ ID NO:22), GSESGIFTNTKESS (SEQ ID NO:23), DEAGSEADHEGTHST (SEQ ID NO:24), SESGIFTNTKESS (SEQ ID NO:25), DEAGSEADHEGTHSTKR (SEQ ID NO:26), NRGDSTFES (SEQ ID NO:27), FLAEGGGV (SEQ ID NO:28), SYNRGDSTFES (SEQ ID NO:29), NRGDSTFESKS (SEQ ID NO:30), STFESKSY (SEQ ID NO:31), DFLAEGG (SEQ ID NO:32), EGDFLAEGGGV (SEQ ID NO:33), EGDFLAEGGG (SEQ ID NO:34), MADEAGSEADHEGTHST (SEQ ID NO:35), DFLAEGGGV (SEQ ID NO:36), DSTFESKSY (SEQ ID NO:37), FTSSTSYNRGDSTFES (SEQ ID NO:38), DSGEGDFLAEGGGV (SEQ ID NO:39), SYKMADEAGSEADHEGTHST (SEQ ID NO:40), DFLAEGGGVR (SEQ ID NO:41), YKMADEAGSEADHEGTHST (SEQ ID NO:42), DFLAEGGG (SEQ ID NO:43), ADSGEGDFLAEGGGV (SEQ ID NO:44), NRGDSTFESKSY (SEQ ID NO:45), GSGSGWSSSRGPY (SEQ ID NO:46), LLGLPGPPDVPDHAAYHPF (SEQ ID NO:47), LDDDLEHQ (SEQ ID NO:48), IGEIKEETT (SEQ ID NO:49), LDDDLEHQGGHVLDHGH (SEQ ID NO:50), SKETIEQEKQAGES (SEQ ID NO:51), KETIEQEKQAGES (SEQ ID NO:52), ETIEQEKQAGES (SEQ ID NO:53), and GPPASSPAPAPK (SEQ ID NO:54)

4. The method according to claim 2, wherein the panel comprise 6 or more peptides derived from the polypeptides A1AT, APO-L1, FGA, ITIH4, KNG-1, and TMSB4.

5. The method according to claim 4, wherein the panel comprises 6 or more peptides selected from the group consisting of EDPQGDAAQKTDT (SEQ ID NO:1), VTEPISAESGEQVER (SEQ ID NO:11), GSESGIFTNTKE (SEQ ID NO:17), SEADHEGTHST (SEQ ID NO:18), SESGIFTNTKE (SEQ ID NO:19), DEAGSEADHEGTH (SEQ ID NO:20), GDFLAEGGGV (SEQ ID NO:21), DEAGSEADHEGT, GSESGIFTNTKESS (SEQ ID NO:23), DEAGSEADHEGTHST (SEQ ID NO:24), SESGIFTNTKESS (SEQ ID NO:25), DEAGSEADHEGTHSTKR (SEQ ID NO:26), NRGDSTFES (SEQ ID NO:27), FLAEGGGV (SEQ ID NO:28), SYNRGDSTFES (SEQ ID NO:29), NRGDSTFESKS (SEQ ID NO:30), STFESKSY (SEQ ID NO:31), DFLAEGG (SEQ ID NO:32), EGDFLAEGGGV (SEQ ID NO:33), EGDFLAEGGG (SEQ ID NO:34), MADEAGSEADHEGTHST (SEQ ID NO:35), DFLAEGGGV (SEQ ID NO:36), DSTFESKSY (SEQ ID NO:37), FTSSTSYNRGDSTFES (SEQ ID NO:38), DSGEGDFLAEGGGV (SEQ ID NO:39), SYKMADEAGSEADHEGTHST (SEQ ID NO:40), DFLAEGGGVR (SEQ ID NO:41), YKMADEAGSEADHEGTHST (SEQ ID NO:42), DFLAEGGG (SEQ ID NO:43), ADSGEGDFLAEGGGV (SEQ ID NO:44), NRGDSTFESKSY (SEQ ID NO:45), LLGLPGPPDVPDHAAYHPF (SEQ ID NO:47), LDDDLEHQ (SEQ ID NO:48), IGEIKEETT (SEQ ID NO:49), LDDDLEHQGGHVLDHGH (SEQ ID NO:50), SKETIEQEKQAGES (SEQ ID NO:51), KETIEQEKQAGES (SEQ ID NO:52), and ETIEQEKQAGES (SEQ ID NO:53).

6. The method according to claim 5, wherein the panel comprises the peptides EDPQGDAAQKTDT (SEQ ID NO:1), VTEPISAESGEQVER (SEQ ID NO:11), GSESGIFTNTKESS (SEQ ID NO:23), GSESGIFTNTKE (SEQ ID NO:17), SESGIFTNTKE (SEQ ID NO:19), SYKMADEAGSEADHEGTHST (SEQ ID NO:40), DEAGSEADHEGTHST (SEQ ID NO:24), DEAGSEADHEGT, SEADHEGTHST (SEQ ID NO:18), ADSGEGDFLAEGGGV (SEQ ID NO:44), DSGEGDFLAEGGGV (SEQ ID NO:39), DFLAEGGGV (SEQ ID NO:36), NRGDSTFESKSY (SEQ ID NO:45), NRGDSTFES (SEQ ID NO:27), DSTFESKSY (SEQ ID NO:37), LLGLPGPPDVPDHAAYHPF (SEQ ID NO:47), LDDDLEHQ (SEQ ID NO:48), IGEIKEETT (SEQ ID NO:49), and SKETIEQEKQAGES (SEQ ID NO:51).

7. The method according to claim 1, wherein the blood sample is a plasma sample.

8. The method according to claim 1, wherein the sample is obtained from the subject at or before gestational week 34.

9. The method according to claim 8, wherein the sample is obtained from the subject at or before gestational week 25.

10. The method according to claim 1, wherein obtaining a preeclampsia peptide representation comprises:
measuring the amount of each peptide of a preeclampsia peptide panel in the sample; and
evaluating the abundance of peptides to arrive at a preeclampsia peptide representation.

11. The method according to claim 10, wherein the measuring comprises mass spectrometry.

12. The method according to claim 1, wherein the providing comprises:
comparing the preeclampsia peptide representation to a reference, and
providing a diagnosis or prognosis based on the comparison.

13. A method for providing a preeclampsia peptide representation for a subject, comprising:
obtaining a blood sample from the subject;
measuring the amount of each peptide of a preeclampsia peptide panel in the sample;
evaluating the abundance of peptides to arrive at a preeclampsia peptide representation; and
providing a report comprising the preeclampsia peptide representation.

14. The method according to claim 13, wherein the measuring comprises mass spectrometry.

15. The method according to claim 14, wherein the evaluating comprises:
obtaining an abundance score for each peptide, comprising:
summing the amount of each preeclampsia peptide across MS fractions, and
normalizing to the sum of the amounts of all preeclampsia peptides across all MS fractions; and
analyzing the abundance scores by predictive analysis of microarrays (PAM) to arrive at the preeclampsia peptide representation.

16. The method according to claim 1, wherein the sample is obtained from the subject at or before gestational week 34.

17. The method according to claim 3, wherein the sample is obtained from the subject at or before gestational week 25.

18. A kit for obtaining a preeclampsia peptide representation for a panel of preeclampsia peptides, comprising:
isotope-labeled peptides corresponding to the peptides of the panel.
